SEQUENCE LISTING (20 sequences)

SEQ ID NO: 1 - protein, 512 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSS

SEQ ID NO: 2 - protein, 512 aa, Artificial
  Lama
VQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 3 - protein, 384 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSS

SEQ ID NO: 4 - protein, 384 aa, Artificial
  Lama
VQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 5 - protein, 522 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSS

SEQ ID NO: 6 - protein, 522 aa, Artificial
  Lama
VQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 7 - protein, 522 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSS

SEQ ID NO: 8 - protein, 522 aa, Artificial
  Lama
VQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 9 - protein, 381 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCSGLGYDYWGQGTLVTVSS

SEQ ID NO: 10 - protein, 381 aa, Artificial
  Lama
VQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCSGLGYDYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 11 - protein, 512 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 12 - protein, 512 aa, Artificial
  Lama
VQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 13 - protein, 384 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 14 - protein, 384 aa, Artificial
  Lama
VQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 15 - protein, 522 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 16 - protein, 522 aa, Artificial
  Lama
VQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 17 - protein, 522 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 18 - protein, 522 aa, Artificial
  Lama
VQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 19 - protein, 381 aa, Artificial
  Lama
VQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCSGLGYDYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 20 - protein, 381 aa, Artificial
  Lama
VQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCSGLGYDYWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

(19) United States
(12) Patent Application Publication
(10) Pub. No.: US 2014/0093499 A1
(43) Pub. Date: Apr. 3, 2014

(54) PHARMACEUTICAL COMBINATIONS COMPRISING DUAL ANGIOPOIETIN-2 / DLL4 BINDERS AND ANTI-VEGF AGENTS

(71) Applicants: Andreas GSCHWIND, Ingelheim am Rhein (DE); Anke BAUM, Hinterbruehl (AT)

(72) Inventors: Andreas GSCHWIND, Ingelheim am Rhein (DE); Anke BAUM, Hinterbruehl (AT)

(73) Assignee: BOEHRINGER INGELHEIM INTERNATIONAL GMBH, Ingelheim am Rhein (DE)

(21) Appl. No.: 14/037,463

(22) Filed: Sep. 26, 2013

(30) Foreign Application Priority Data
Sep. 28, 2012 (EP) 12186696.6

(51) Int. Cl. A61K 39/395; A61K 38/17; A61K 31/496

(52) U.S. Cl. CPC A61K 39/3955; A61K 31/496; A61K 38/1761
USPC 424/133.1; 514/18.9

(57) ABSTRACT

The present invention relates to pharmaceutical combinations comprising dual Angiopoietin-2/Dll4 binders and anti-VEGF agents for use in treating diseases like cancer and ocular diseases.

### FIELD OF INVENTION

The present invention relates to pharmaceutical combinations comprising dual Angiopoietin-2/Dll4 binders and anti-VEGF agents for use in treating diseases like cancer, ocular diseases and others.

### BACKGROUND OF INVENTION

When tumors reach a critical size of approximately 1 mm³ they become dependent on angiogenesis for maintaining blood supply with oxygen and nutrients to allow for further growth. As summarized in US 2008/0014196, angiogenesis is implicated in the pathogenesis of a number of disorders, including solid tumors and metastasis.

In the case of tumor growth, angiogenesis appears to be crucial for the transition from hyperplasia to neoplasia, and for providing nourishment for the growth and metastasis of the tumor (Folkman et al., Nature 339-58, 1989), which allows the tumor cells to acquire a growth advantage compared to the normal cells. Therefore, anti-angiogenesis therapies have become an important treatment option for several types of tumors. These therapies have focused on blocking the VEGF pathway (Ferrara et al., Nat Rev Drug Discov. 2004 May; 3(5):391-400) by neutralizing VEGF (Avastin) or its receptors (Sutent and Sorafinib).

As described in e.g. US2008/0014196 and WO2008/101985, angiogenesis is implicated in the pathogenesis of a number of disorders, including solid tumors and metastasis as well as eye diseases. One of the most important pro-angiogenic factors is vascular endothelial growth factor (VEGF), also termed VEGF-A or vascular permeability factor (VPF). VEGF belongs to a gene family that includes placenta growth factor (PlGF), VEGF-B, VEGF-C, VEGF-D, VEGF-E and VEGF-F. Alternative splicing of mRNA of a single gene of human VEGF results in at least six isoforms (VEGF121, VEGF145, VEGF165, VEGF183, VEGF189, and VEGF206), VEGF165 being the most abundant isoform.

Two VEGF tyrosine kinase receptors (VEGFR) have been identified that interact with VEGF, i.e. VEGFR-1 (also known as Flt-1) and VEGFR-2 (also known as KDR or FIK-1). VEGFR-1 has the highest affinity for VEGF, while VEGFR-2 has a somewhat lower affinity for VEGF. Ferrara (Endocrine Rev. 2004, 25: 581-611) provide a detailed description of VEGF, the interaction with its receptors and its function in normal and pathological processes can be found in Hoeben et al. Pharmacol. Rev. 2004, 56: 549-580.

VEGF has been reported to be a pivotal regulator of both normal and abnormal angiogenesis (Ferrara and Davis-Smyth, Endocrine Rev. 1997, 18: 4-25; Ferrara J. Mol. Med. 1999, 77: 527-543). Compared to other growth factors that contribute to the processes of vascular formation, VEGF is unique in its high specificity for endothelial cells within the vascular system.

VEGF mRNA is overexpressed by the majority of human tumors. In the case of tumor growth, angiogenesis appears to be crucial for the transition from hyperplasia to neoplasia, and for providing nourishment for the growth and metastasis of the tumor (Folkman et al., 1989, Nature 339-58), which allows the tumor cells to acquire a growth advantage compared to the normal cells. Therefore, anti-angiogenesis therapies have become an important treatment option for several types of tumors. These therapies have focused on blocking the VEGF pathway (Ferrara et al., Nat Rev Drug Discov. 2004 May; 3(5): 391-400.

The elucidation of VEGF and its role in angiogenesis and different processes has provided a potential new target of therapeutic intervention. The function of VEGF has been inhibited by small molecules that block or prevent activation of VEGF receptor tyrosine kinases (Schlaeppi and Wood, 1999, Cancer Metastasis Rev., 18: 473-481) and consequently interfere with the VEGF receptor signal transduction pathway. Cytotoxic conjugates containing bacterial or plant toxins can inhibit the stimulating effect of VEGF on tumor angiogenesis. VEGF-DT385 toxin conjugates (diphtheria toxin domains fused or chemically conjugated to VEGF165), for example, efficiently inhibit tumor growth in vivo. Tumor growth inhibition could also be achieved by delivering a Flk-1 mutant or soluble VEGF receptors by a retrovirus.

VEGF-neutralizing antibodies, such as A4.6.1 and MV833, have been developed to block VEGF from binding to its receptors and have shown preclinical antitumor activity (Kim et al. Nature 1993, 362: 841-844; Folkman Nat. Med. 1995, 1: 27-31; Presta et al. Cancer Res. 1997, 57: 4593-4599; Kanai et al. Int. J. Cancer 1998, 77: 933-936; Ferrara and Alitalo Nat. Med. 1999, 5: 1359-1364; 320, 340. For a review of therapeutic anti-VEGF approaches trials, see Campochiaro and Hackett, Oncogene 2003, 22: 6537-6548).

Most clinical experience has been obtained with A4.6.1, also called bevacizumab (Avastin®; Genentech, San Francisco, Calif.). Recent studies in mice have shown, that Angiopoietin2 (Ang2), a ligand of the Tie2 receptor, controls vascular re-modeling by enabling the functions of other angiogenic factors, such as VEGF. Ang2 is primarily expressed by endothelial cells, strongly induced by hypoxia and other angiogenic factors and has been demonstrated to regulate tumor vessel plasticity, allowing vessels to respond to VEGF and FGF2 (Augustin et al., Nat Rev Mol Cell Biol. 2009 March; 10(3):165-77). Consistent with this role, the deletion or inhibition of Ang2 results in reduced angiogenesis (Falcón et al., Am J Pathol. 2009 November; 175(5):2159-70). Elevated Ang2 serum concentrations have been reported for patients with colorectal cancer, NSCLC and melanoma (Goede et al., Br J. Cancer. 2010 Oct. 26; 103(9):1407-14; Park et al., Chest. 2007 July; 132(1): 200-6; Helfrich et al., Clin Cancer Res. 2009 Feb. 15; 15(4):1384-92). In CRC cancer Ang2 serum levels correlate with therapeutic response to anti-VEGF therapy.

The Ang-Tie system consists of 2 receptors (Tie1 and Tie2) and 3 ligands (Ang1, Ang2 and Ang4) (Augustin et al., Nat Rev Mol Cell Biol. 2009 March; 10(3):165-77). Tie2, Ang1 and Ang2 are the best studied members of this family, Tie1 is an orphan receptor and the role of Ang4 for vascular remodelling still needs to be defined. Ang2 and Ang1 mediate opposing functions upon Tie2 binding and activation. Ang2-mediated Tie2 activation results in endothelial cell activation, pericyte dissociation, vessel leakage and induction of vessel sprouting. In contrast to Ang2, Ang1 signalling maintains vessel integrity by recruitment of pericytes, thereby maintaining endothelial cell quiescence.

Ang2 is a secreted, 66 kDa ligand for the Tie2 receptor tyrosine kinase (Augustin et al., Nat Rev Mol Cell Biol. 2009 March; 10(3):165-77). Ang2 consists of an N-terminal coiled-coil domain and a C-terminal fibrinogen-like domain, the latter is required for Tie2 interaction. Ang2 is primarily expressed by endothelial cells and strongly induced by hypoxia and other angiogenic factors, including VEGF. Tie2 is found on endothelial cells, haematopoietic stem cells and tumor cells. Ang2-Tie2 has been demonstrated to regulate tumor vessel plasticity, allowing vessels to respond to VEGF and FGF2.

In vitro Ang2 has been shown to act as a modest mitogen, chemo-attractant and inducer of tube formation in human umbilical vein endothelial cells (HUVEC). Ang2 induces tyrosine phosphorylation of ectopically expressed Tie2 in fibroblasts and promotes downstream signaling events, such as phosphorylation of ERK-MAPK, AKT and FAK in HUVEC. An antagonistic role of Ang2 in Ang1-induced endothelial cell responses has been described.

Ang2 deficiency has been shown to result in a profound lymphatic patterning defect in mice. Although the loss of Ang2 is dispensable for embryonic vascular development, Ang2-deficient mice have persistent vascular defects in the retina and kidney. Together with the dynamic pattern of Ang2 expression at sites of angiogenesis (for example ovary), these findings indicate that Ang2 controls vascular re-modeling by enabling the functions of other angiogenic factors, such as VEGF.

The Ang2-Tie2 system exerts crucial roles during the angiogenic switch and later stages of tumor angiogenesis. Ang2 expression is strongly up-regulated in the tumor-associated endothelium. Reduced growth of tumors has been observed when implanted into Ang2-deficient mice, especially during early stages of tumor growth. Therapeutic blocking of Ang2 with Ang2 mAbs has shown broad efficacy in a variety of tumor xenograft models.

The Notch signalling pathway is important for cell-cell communication, which involves gene regulation mechanisms that control multiple cell differentiation processes during embryonic development and in adult organisms. Notch signalling is dysregulated in many cancers, e.g. in T-cell acute lymphoblastic leukemia and in solid tumors (Sharma et al. 2007, Cell Cycle 6 (8): 927-30; Shih et al., Cancer Res. 2007 Mar. 1; 67(5): 1879-82).

Dll4 (or Delta like 4 or delta-like ligand 4) is a member of the Delta family of Notch ligands. The extracellular domain of Dll4 is composed of an N-terminal domain, a Delta/Serrate/Lag-2 (DSL) domain, and a tandem of eight epidermal growth factor (EGF)-like repeats. Generally, the EGF domains are recognized as comprising amino acid residues 218-251 (EGF-1; domain 1), 252-282 (EGF-2; domain 2), 284-322 (EGF-3; domain 3), 324-360 (EGF-4; domain 4), and 362-400 (EGF-5; domain 5), with the DSL domain at about amino acid residues 173-217 and the N-terminal domain at about amino acid residues 27-172 of hDll4 (WO 2008/076379).

It has been reported that Dll4 exhibits highly selective expression by vascular endothelium, in particular in arterial endothelium (Shutter et al. (2000) Genes Develop. 14: 1313-1318). Recent studies in mice have shown that Dll4 is induced by VEGF and is a negative feedback regulator that restrains vascular sprouting and branching. Consistent with this role, the deletion or inhibition of Dll4 results in excessive angiogenesis (Scehnet et al., Blood. 2007 Jun. 1; 109(11):4753-60). This unrestrained angiogenesis paradoxically decreases tumor growth due to the formation of non-productive vasculature, even in tumors resistant to anti-VEGF therapies (Thurston et al., Nat Rev Cancer. 2007 May; 7(5):327-31; WO 2007/070671; Noguera-Troise et al., Nature. 2006 Dec. 21; 444(7122)). Furthermore, the combined inhibition of VEGF and Dll4 is shown to provide superior anti-tumor activity compared to anti-VEGF alone in xenograft models of multiple tumor types (Noguera-Troise et al., Nature. 2006 Dec. 21; 444(7122):1032-7; Ridgway et al., Nature. 2006 Dec. 21; 444(7122):1083-7).

Due to these results, Dll4 is being considered a promising target for cancer therapy, and several biological compounds that target Dll4 are in (pre-)clinical development have been described: REGN-421 (═SAR153192; Regeneron, Sanofi-Aventis; WO2008076379), OPM-21M18 (OncoMed; Hoey et al., Cell Stem Cell. 2009 Aug. 7; 5(2):168-77) and MED10639 (MedImmune LLC, AstraZeneca; Jenkins et al., Mol Cancer Ther. 2012 August; 11(8):1650-60) fully human Dll4 antibodies; YW152F (Genentech), a humanized Dll4 antibody (Ridgway et al., Nature. 2006 Dec. 21; 444(7122):1083-7); Dll4-Fc (Regeneron, Sanofi-Aventis), a recombinant fusion protein composed of the extracellular region of Dll4 and the Fc region of human IgG1 (Noguera-Troise et al., Nature. 2006 Dec. 21; 444(7122)).

However, the state-of-the art monoclonal antibodies (MAbs) and fusion proteins have several shortcomings in view of their therapeutic application: To prevent their degradation, they must be stored at near freezing temperatures. Also, since they are quickly digested in the gut, they are not suited for oral administration. Another major restriction of mAbs for cancer therapy is poor tumor tissue penetration, which results in low concentrations and a lack of targeting of all cells in a tumor. Die most severe shortcoming of the prior art antibodies in this field is their limited clinical efficacy.

### SUMMARY OF THE INVENTION

Shortcomings of currently available anti-angiogenesis therapies have been limited efficacy. It has thus been an object of the present invention to improve anti-angiogenesis therapy.

Another object of the present invention is to improve anti-angiogenesis therapy in the context of intrinsic or acquired resistance to therapy.

It is a further object of the present invention to provide such therapies, which are well-tolerable for the patient.

The present inventors have found that pharmaceutical combinations comprising dual anti-Ang2/anti-Dll4 binders and anti-VEGF agents have a higher anti-cancer efficacy than the individual agents alone, which can be used in human therapy.

Based on this finding the present invention provides novel pharmaceutical combinations comprising dual anti-Ang2/anti-Dll4 binders and anti-VEGF agents, especially suited for the treatment of cancer and of ocular diseases.

It is a further beneficial feature of the combinations according to the present invention that resistance to therapy can be mediated through several redundant angiogenic signal transduction pathways.

In another aspect, the present invention also relates to dual anti-Ang2/anti-Dll4 binders for use in the treatment of cancer in combination with anti-VEGF agents.

In another aspect, the present invention relates to a method of treatment of cancer, comprising administration of a therapeutically effective amount of a dual anti-Ang2/anti-Dll4 binder to a patient in need thereof, and furthermore comprising administration of a therapeutically effective amount of an anti-VEGF agent to the same patient within 72 hours before or after administration of said dual anti-Ang2/anti-Dll4 binder.

### BRIEF DESCRIPTION OF THE FIGURES

FIG. 1 shows NCl-H1975 tumor growth kinetics. NCl-H1975 tumor-bearing mice were treated with Bevacizumab, BIBF 1120, BI-1, the combination of Bevacizumab and BI-1, the combination of BIBF 1120 and BI-1 or with the vehicle only. Median tumor volumes are plotted over time. Day 1 was the first day, day 14 the last day of the experiment.

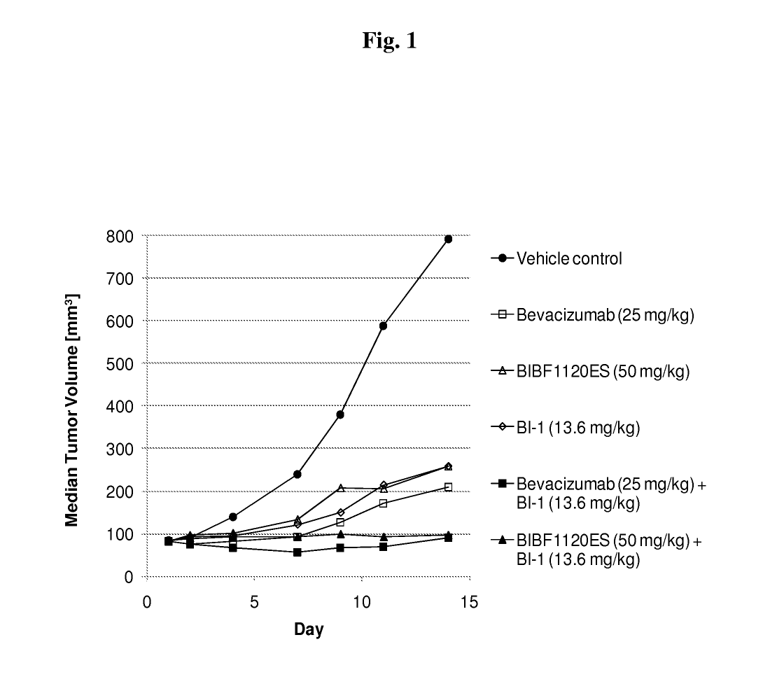

FIG. 2 shows absolute tumor volumes on day 19. NCl-H1975 tumor-bearing mice were treated with Bevacizumab, BIBF 1120, BI-1, the combination of Bevacizumab and BI-1, the combination of BIBF 1120 and BI-1 or with the vehicle only. Individual absolute tumor volumes are plotted at day 14. Each symbol represents an individual tumor. The horizontal lines represent the median tumor volumes.

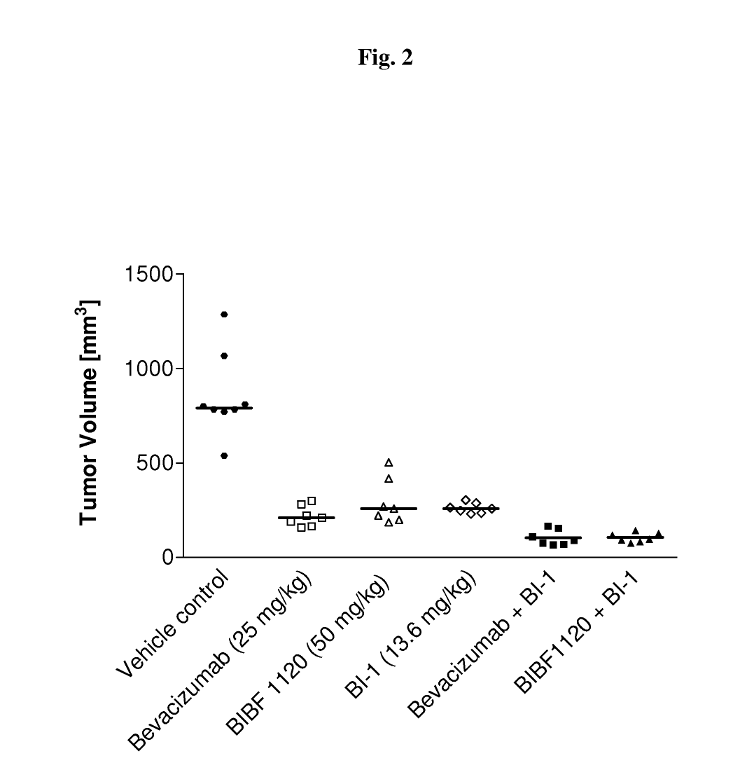

FIG. 3 shows the change of body weight over time. NCl-H1975 tumor-bearing mice were treated with Bevacizumab, BIBF 1120, BI-1, the combination of Bevacizumab and BI-1, the combination of BIBF 1120 and BI-1 or with the vehicle only. Median changes of body weight are plotted over time. Day 1 was the first day, day 14 the last day of the experiment.

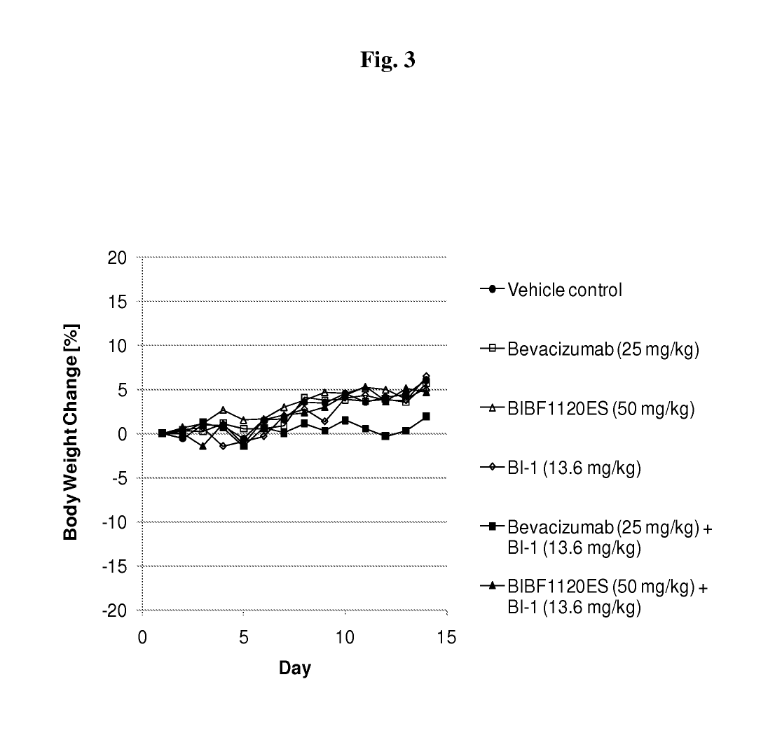

FIG. 4 shows CXF 243 tumor growth kinetics. CXF 243 tumor-bearing mice were treated with BI-1, BIBF 1120, the combination of BI-1 and BIBF 1120 or with the vehicle only. Median tumor volumes are plotted over time.

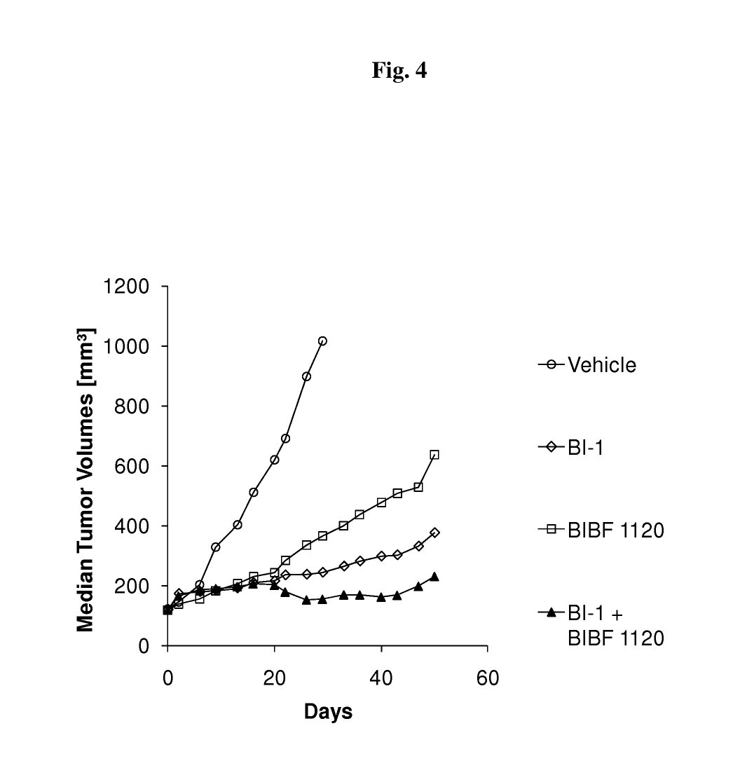

FIG. 5 shows LXFE 211 tumor growth kinetics. LXFE 211 tumor-bearing mice were treated with BI-1, Bevacizumab, the combination of BI-1 and Bevacizumab or with the vehicle only. Median tumor volumes are plotted over time.

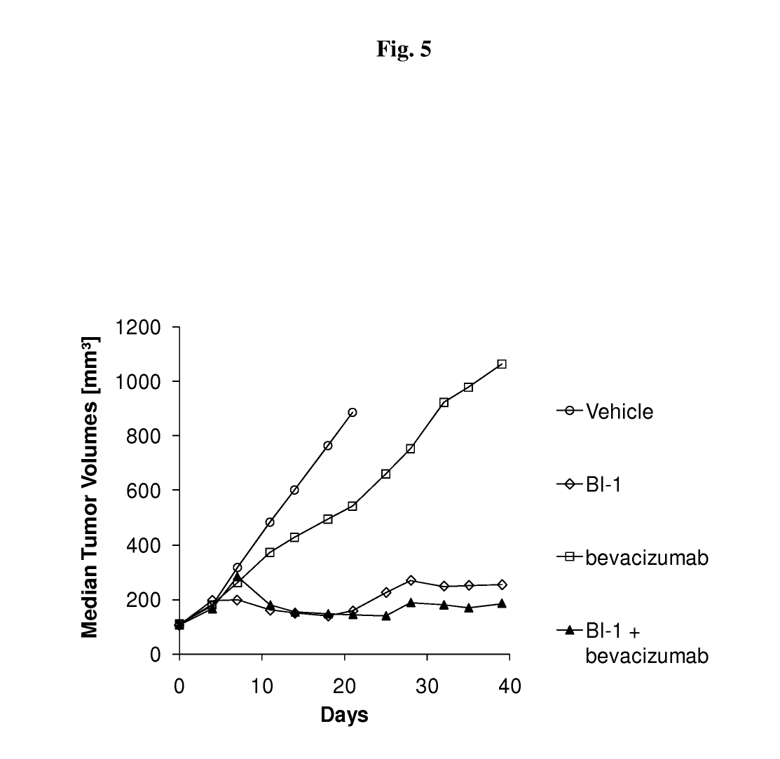

FIG. 6 shows LXFE 211 tumor growth kinetics. LXFE 211 tumor-bearing mice were treated with BI-1, BIBF 1120, the combination of BI-1 and BIBF 1120 or with the vehicle only. Median tumor volumes are plotted over time.

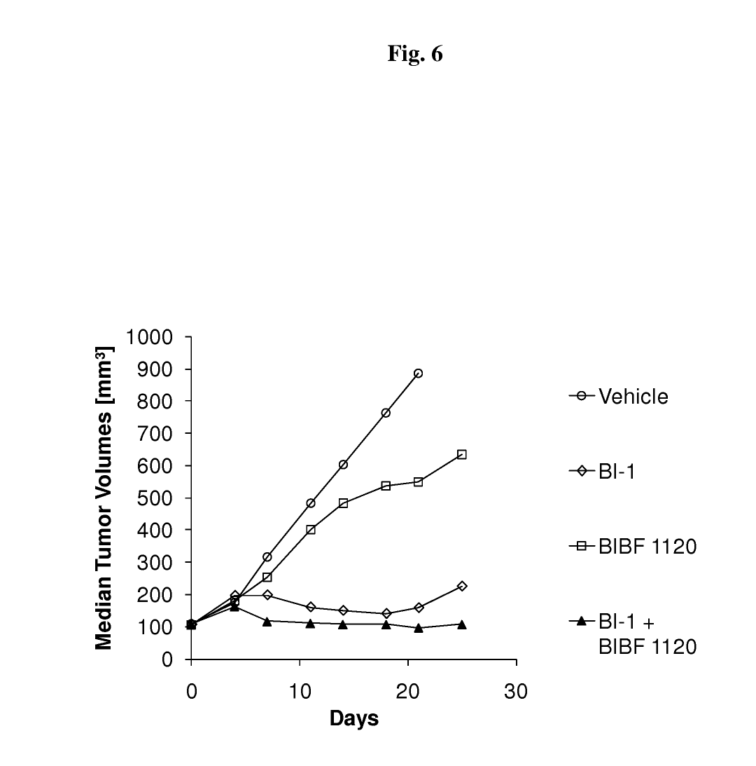

FIG. 7 shows LXFE 1422 tumor growth kinetics. LXFE 1422 tumor-bearing mice were treated with BI-1, Bevacizumab, the combination of BI-1 and Bevacizumab or with the vehicle only. Median tumor volumes are plotted over time.

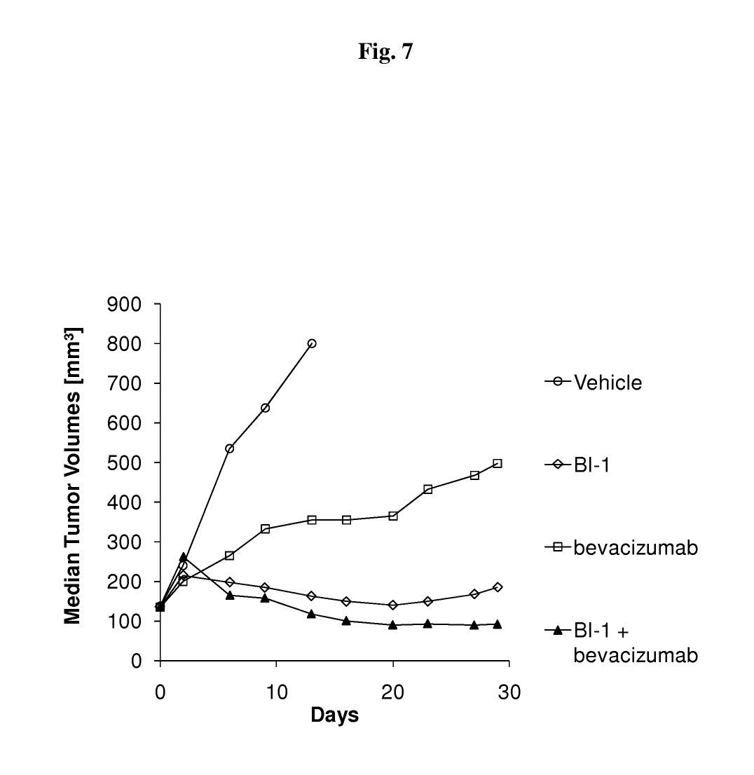

FIG. 8 shows LXFE 1422 tumor growth kinetics. LXFE 1422 tumor-bearing mice were treated with BI-1, BIBF 1120, the combination of BI-1 and BIBF 1120 or with the vehicle only. Median tumor volumes are plotted over time.

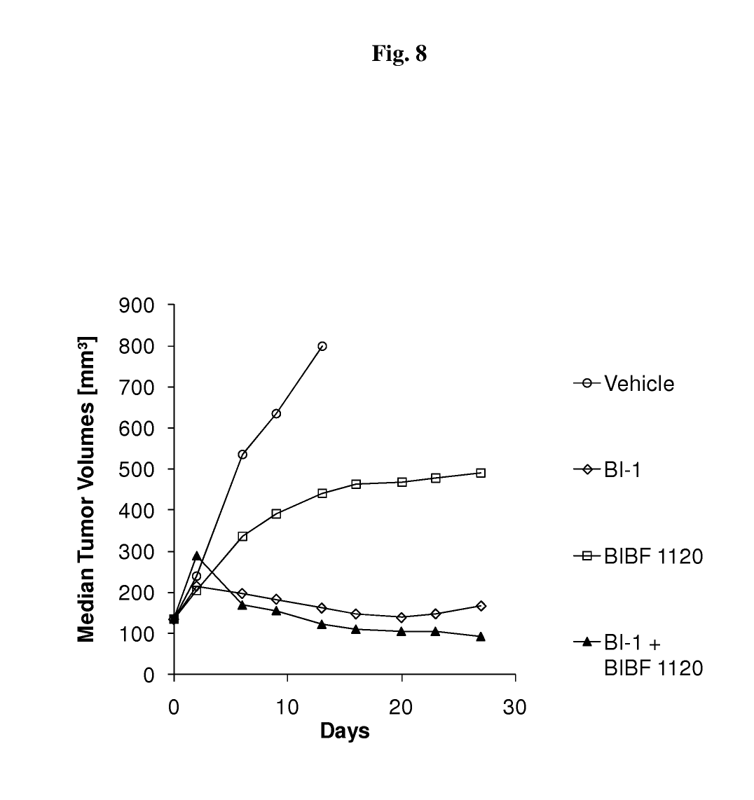

FIG. 9 shows MAXF 401 tumor growth kinetics. MAXF 401 tumor-bearing mice were treated with BI-1, Bevacizumab, the combination of BI-1 and Bevacizumab or with the vehicle only. Median tumor volumes are plotted over time.

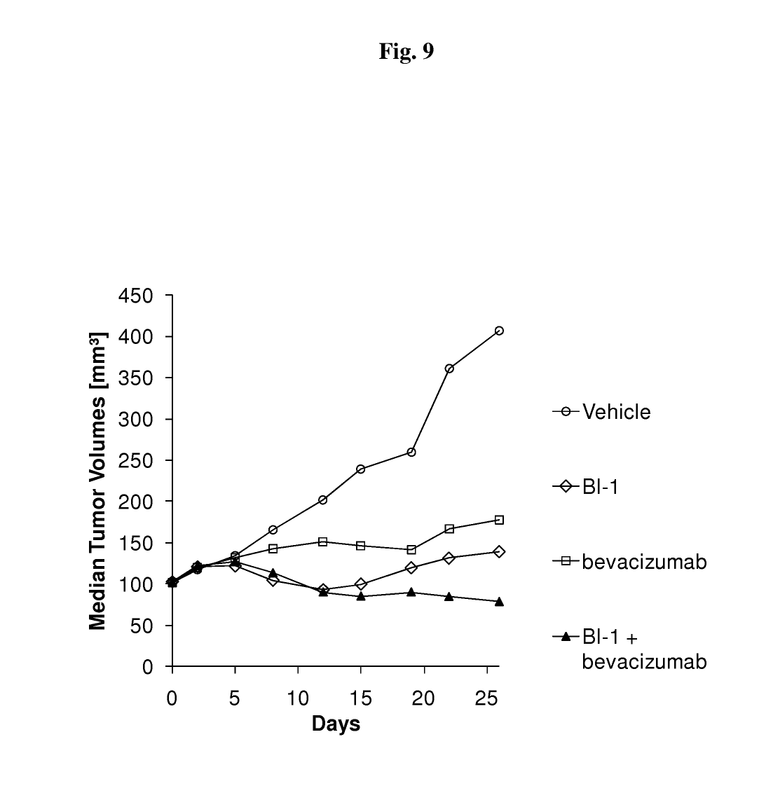

FIG. 10 shows MAXF 401 tumor growth kinetics. MAXF 401 tumor-bearing mice were treated with BI-1, BIBF 1120, the combination of BI-1 and BIBF 1120 or with the vehicle only. Median tumor volumes are plotted over time.

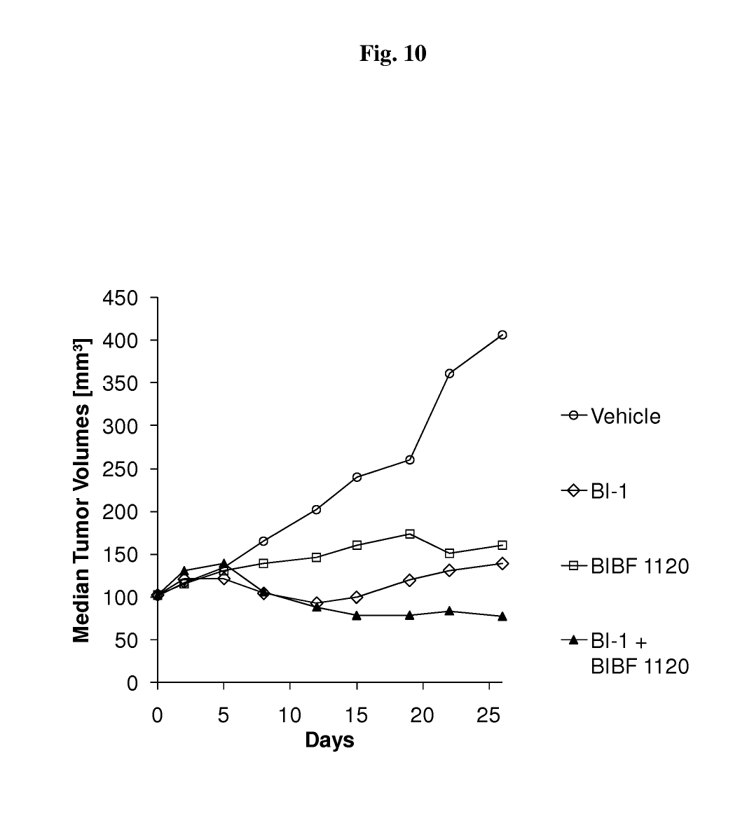

FIG. 11 shows OVXF 1353 tumor growth kinetics. OVXF 1353 tumor-bearing mice were treated with BI-1, BIBF 1120, the combination of BI-1 and BIBF 1120 or with the vehicle only. Median tumor volumes are plotted over time.

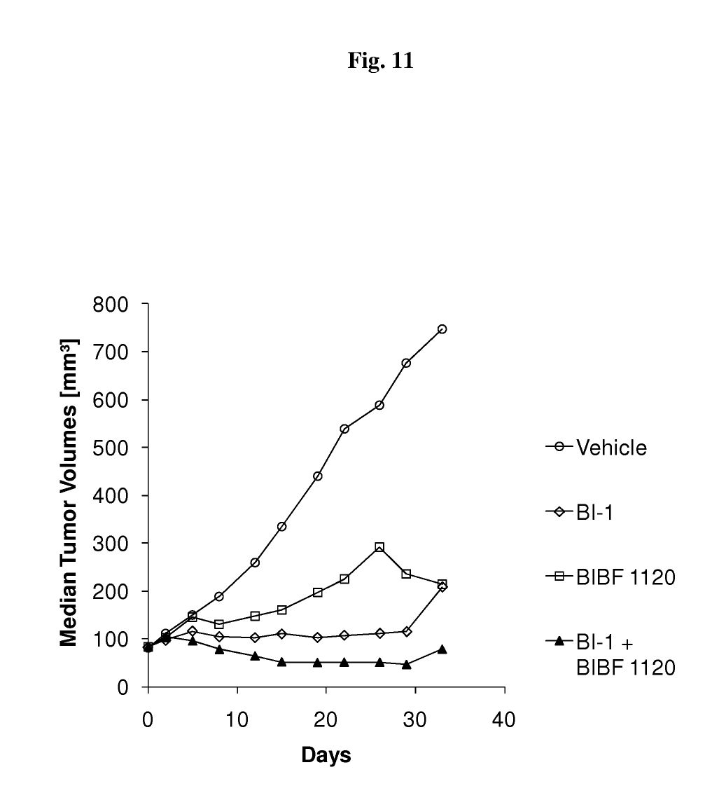

FIG. 12 shows PAXF 546 tumor growth kinetics. PAXF 546 tumor-bearing mice were treated with BI-1, Bevacizumab, the combination of BI-1 and Bevacizumab or with the vehicle only. Median tumor volumes are plotted over time.

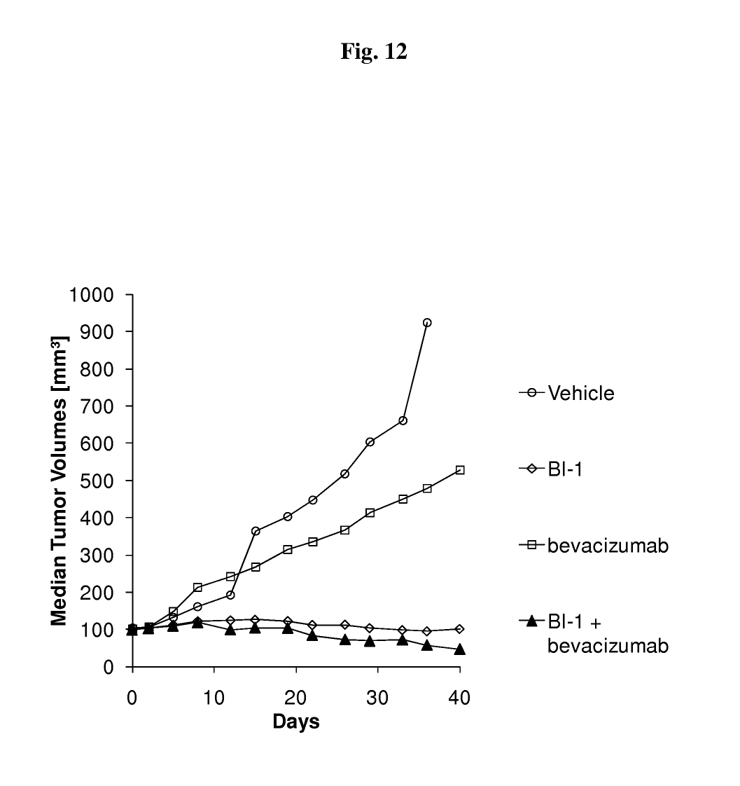

FIG. 13 shows PAXF 546 tumor growth kinetics. PAXF 546 tumor-bearing mice were treated with BI-1, BIBF 1120, the combination of BI-1 and BIBF 1120 or with the vehicle only. Median tumor volumes are plotted over time.

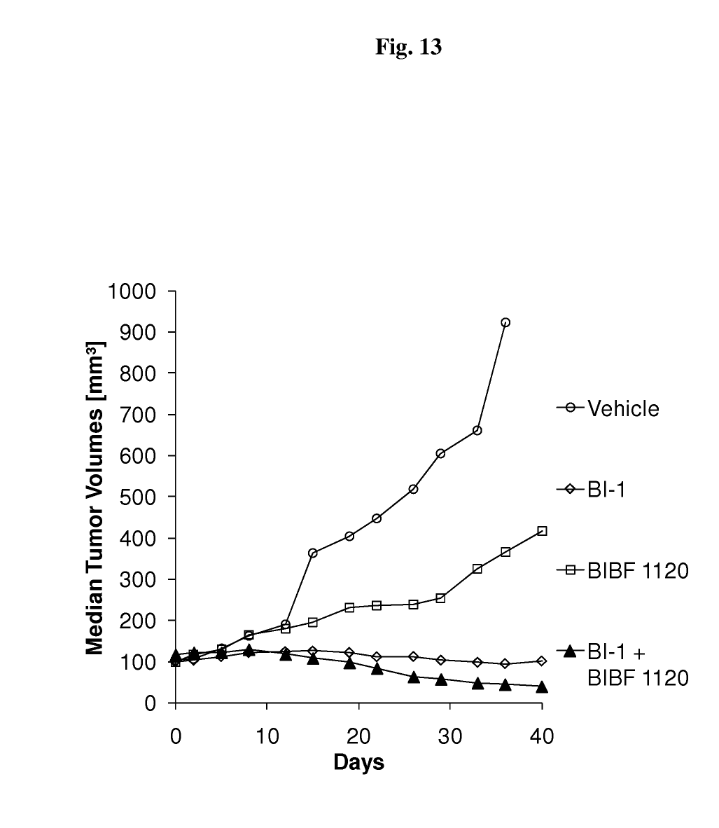

FIG. 14 shows RXF 1220 tumor growth kinetics. RXF 1220 tumor-bearing mice were treated with BI-1, Sunitinib, the combination of BI-1 and Sunitinib or with the vehicle only. Median tumor volumes are plotted over time.

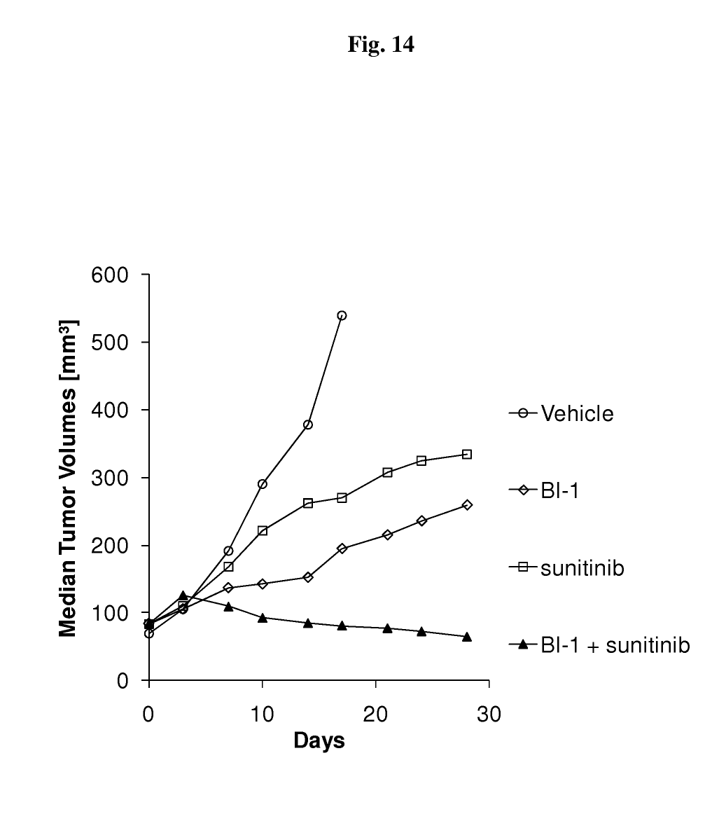

### DETAILED DESCRIPTION OF THE INVENTION

“Pharmaceutical combinations” as used herein refer to two or more different pharmaceutically-active substances, which are intended to produce a specific therapeutic effect in a patient when applied together to said patient, i.e. one or more dual anti-Ang2/anti-Dll4 binders and one or more anti-VEGF agents in the context of the present invention. “Applied together” herein means either subsequent application or simultaneous application.

In one embodiment, the dual anti-Ang2/anti-Dll4 binder is to be administered at any time point between 6 months and 1 week prior to administration of the anti-VEGF agent. In preferred embodiments, the dual anti-Ang2/anti-Dll4 binder is to be administered at any time point between 3 months and 1 week, six weeks and 1 week, 1 month and 1 week, 3 weeks and 1 week, and 2 weeks and 1 week prior to administration of the anti-VEGF agent. In one embodiment, the dual anti-Ang2/anti-Dll4 binder is to be administered at any time point between 1 week and 0 days prior to administration of the anti-VEGF agent.

Of course, it is also within the scope of the invention that the anti-VEGF agent is administered prior to the dual anti-Ang2/anti-Dll4 binder. Hence, the aforementioned embodiment applies to this alternative embodiment, mutatis mutandis.

The administration of the dual anti-Ang2/anti-Dll4 binder concurrently with the anti-VEGF agent mean that both medicaments are administered at the same time. This can be achieved by having both dual anti-Ang2/anti-Dll4 binder and anti-VEGF agent present in one dose, vial, bag, container, syringe, etc.

A subsequent administration of the dual anti-Ang2/anti-Dll4 binder and anti-VEGF agent means that the anti-VEGF agent is administered shortly after the dual anti-Ang2/anti-Dll4 binders or vice versa. Shortly includes 1, 2, 3, 4, 5, 10, 20, 30, 45, 60 minutes, 2, 3, 4, 5, 6, 7, 8, 9, 10, 12, 14, 16, 18, 20, 22 or 24 hours.

“Patient” herein refers to mammals, particularly humans.

“Dual anti-Ang2/anti-Dll4 binders” as used herein refers to any peptide-based molecule capable of inhibiting the pro-angiogenic activity of both Ang2 and Dll4 by at least 80%. Suitable dual anti-Ang2/anti-Dll4 binders preferably comprise separate binding regions for each Ang2 and Dll4. Suitable dual anti-Ang2/anti-Dll4 binders can be formed by any bi-specific binding molecule known in the art, for instance cross-linked Fabs, cross-linked scFvs, dual-specific IgGs, crossmabs, Fcabs, zybodies, surrobodies, single light chain (sLC) antibodies, DARTs, Nanobodies®, domain antibodies (dAbs), DARPins. In a specific embodiment the dual anti-Ang2/anti-Dll4 binders are Nanobodies®. In preferred embodiments the dual anti-Ang2/anti-Dll4 binders are provided with means for prolonging their half-life in the body. Suitable means for this purpose are for instance human Fc regions or serum albumin molecules fused to the dual anti-Ang2/anti-Dll4 binders. Other suitable means, which are preferred herein, are further binding regions comprised by the dual anti-Ang2/anti-Dll4 binders, which bind to serum albumin. Particularly preferred are such further binding regions, which bind to human albumin-11 (Alb11). Suitable dual anti-Ang2/anti-Dll4 binders can be found in co-pending PCT application PCT/EP2012/055897. In preferred embodiments of the present invention the dual anti-Ang2/anti-Dll4 binders are selected from a binding molecule according to any of SeqID No: 1-20.

“BI-1” is a dual anti-Ang2/anti-Dll4 Nanobody® binder according to SeqID No: 14.

“Anti-VEGF agents” as used herein comprise all pharmaceutically acceptable molecules which inhibit the pro-angiogenic activity of at least VEGF-A, preferably also of VEGF-B and/or VEGF-C and/or VEGF-D. Particularly preferred anti-VEGF agents are bevacizumab, pegaptanib, ranibizumab, aflibercept and PRS-050.

In a preferred embodiment the pharmaceutical combinations herein comprise one or more anti-VEGF agents selected from bevacizumab, pegaptanib, ranibizumab, aflibercept and PRS-050 and one or more dual anti-Ang2/anti-Dll4 binders selected from SeqID No: 1-20.

In another preferred embodiment the pharmaceutical combinations herein comprise a dual anti-Ang2/anti-Dll4 binder according to SeqID No: 14 and bevacizumab.

In another preferred embodiment the pharmaceutical combinations herein comprise a dual anti-Ang2/anti-Dll4 binder according to SeqID No: 15 and bevacizumab.

In another preferred embodiment the pharmaceutical combinations herein comprise a dual anti-Ang2/anti-Dll4 binder according to SeqID No: 16 and bevacizumab.

In another preferred embodiment the pharmaceutical combinations herein comprise a dual anti-Ang2/anti-Dll4 binder according to SeqID No: 17 and bevacizumab.

In another preferred embodiment the pharmaceutical combinations herein comprise a dual anti-Ang2/anti-Dll4 binder according to SeqID No: 18 and bevacizumab.

“Cancer” as used herein generally to all malignant neoplastic diseases. For example, the following cancers may be treated with combinations according to the invention, without being restricted thereto:

brain tumours such as for example acoustic neurinoma, astrocytomas such as pilocytic astrocytomas, fibrillary astrocytoma, protoplasmic astrocytoma, gemistiocytic astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningiomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma.Bone marrow tumours; intestinal cancer such as for example carcinoma of the rectum and colon tumours of the small intestine and duodenum; esophageal cancer or cancer of the esophagus such as squamous cell carcinoma, adenocarcinoma in Barret's esophagus, adenoid cystic carcinoma, small cell carcinoma and lymphoma; eyelid tumours such as basalioma or basal cell carcinoma; pancreatic cancer or carcinoma of the pancreas such as duct cell adenocarcinoma, acinar cell carcinoma, islet cell carcinoma, lymphoma and sarcoma of the pancreas; bladder cancer or carcinoma of the bladder such as superficial and infiltrating transitional cell carcinoma, squamous cell carcinoma and adenocarcinoma; lung cancer (bronchial carcinoma) such as for example small-cell bronchial carcinomas (oat cell carcinomas) and non-small cell bronchial carcinomas (NSCLC) such as squamous cell carcinomas, adenocarcinomas and large-cell bronchial carcinomas; breast cancer such as for example mammary carcinoma such as in situ and infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrioid and serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma such as for example clear cell renal cell carcinoma or hypernephroma or Grawitz's tumour, papillary carcinoma and oncocytoma; oesophageal cancer or carcinoma of the oesophagus; penile cancer; prostate cancer; throat cancer or carcinomas of the pharynx such as for example squamous cell carcinomas of the nasopharynx (nasopharynx carcinomas), oropharynx (oropharynx carcinomas) and hypopharynx carcinomas; retinoblastoma, vagin cancer or vaginal carcinoma and cancers of the vulva including squamous cell carcinomas, adenocarcinomas and in situ carcinomas; malignant melanomas and sarcomas; thyroid carcinomas such as for example papillary, follicular and medullary thyroid carcinoma, as well as anaplastic carcinomas; spinalioma, epiderrmoid carcinoma and basal cell carcinoma of the skin; thymomas, cancer of the urethra including in situ and infiltrating transitional cell carcinoma.
Combinations with Anti-Neoplastic Agents

In preferred embodiments of the invention the pharmaceutical combinations herein further comprise one or more “anti-neoplastic agents”, which term is used herein to refer to a substance producing an anti-neoplastic effect in a tissue, system, animal, mammal, human, or other subject. In particular, in anti-neoplastic therapy, combination therapy with other chemotherapeutic, hormonal, antibody agents as well as surgical and/or radiation treatments other than those mentioned above are envisaged. Combination therapies according to the present invention thus include the administration of dual anti-Ang2/anti-Dll4 binders and anti-VEGF agents as well as optional use of other therapeutic agents including other anti-neoplastic agents. Such combination of agents may be administered together or separately and, when administered separately this may occur simultaneously or sequentially in any order, both close and remote in time.

Depending on the disorder to be treated, the pharmaceutical combinations herein of the invention may be used on its own or in combination with one or more anti-neoplastic agents, in particular selected from DNA damaging, DNA demethylating or tubulin binding agents or therapeutically active compounds that inhibit angiogenesis, signal transduction pathways or mitotic checkpoints in cancer cells or have immunomodulatory function (IMIDs).

The anti-neoplastic agent may be administered simultaneously with, optionally as a component of the same pharmaceutical composition, or before or after administration of the pharmaceutical combinations herein.

In certain embodiments, the anti-neoplastic agent may be, without limitation, one or more inhibitors selected from the group of inhibitors of EGFR family, VEGFR family, IGF-1R, Insulin receptors, AuroraA, AuroraB, PLK and PI3 kinase, FGFR, PDGFR, Raf, KSP or PDK1.

Further examples of anti-neoplastic agents are inhibitors of CDKs, Akt, Src, Bcr-Abl, cKit, cMet/HGF, Her2, Her3, c-Myc, Flt3, HSP90, hedgehog antagonists, inhibitors of JAK/STAT, Mek, mTor, NFkappaB, the proteasome, Rho, an inhibitor of Wnt signaling or Notch signaling or an ubiquitination pathway inhibitor.

Further examples of anti-neoplastic agents are inhibitors of DNA polymerase, topoisomerase II, multityrosine kinase inhibitors, CXCR4 antagonists, IL3RA inhibitors, RAR antagonists, KIR inhibitors, immunotherapeutic vaccines, TUB inhibitors, Hsp70 inducers, IAP family inhibitors, DNA methyltransferase inhibitors, TNF inhibitors, ErbB1 receptor tyrosine kinase inhibitors, multikinase inhibitors, JAK2 inhibitors, RR inhibitors, apoptosis inducers, HGPRTase inhibitors, histamine H2 receptor antagonists and CD25 receptor agnosists.

Examples for Aurora inhibitors are, without limitation, PHA-739358, AZD-1152, AT-9283, CYC-116, R-763, VX-667, MLN-8045, PF-3814735, SNS-314, VX-689, GSK-1070916, TTP-607, PHA-680626, MLN-8237, B1847325 and ENMD-2076.

Examples for PLK inhibitor are GSK-461364, B12536 and B16727.

Examples for raf inhibitors are BAY-73-4506 (also a VEGFR inhibitor), PLX-4032, RAF-265 (also a VEGFR inhibitor), sorafenib (also a VEGFR inhibitor), XL-281, Nevavar (also an inhibitor of the VEGFR) and PLX4032.

Examples for KSP inhibitors are ispinesib, ARRY-520, AZD-4877, CK-1122697, GSK-246053A, GSK-923295, MK-0731, SB-743921, LY-2523355, and EMD-534085.

Examples for a src and/or bcr-abl inhibitors are dasatinib, AZD-0530, bosutinib, XL-228 (also an IGF-1R inhibitor), nilotinib (also a PDGFR and cKit inhibitor), imatinib (also a cKit inhibitor), NS-187, KX2-391, AP-24534 (also an inhibitor of EGFR, FGFR, Tie2, Flt3), KM-80 and LS-104 (also an inhibitor of Flt3, Jak2).

An example for a PDK1 inhibitor is AR-12.

An example for a Rho inhibitor is BA-210.

Examples for PI3 kinase inhibitors are PX-866, PX-867, BEZ-235 (also an mTor inhibitor), XL-147, and XL-765 (also an mTor inhibitor), BGT-226, CDC-0941.

Examples for inhibitors of cMet or HGF are XL-184 (also an inhibitor of VEGFR, cKit, Flt3), PF-2341066, MK-2461, XL-880 (also an inhibitor of VEGFR), MGCD-265 (also an inhibitor of VEGFR, Ron, Tie2), SU-11274, PHA-665752, AMG-102, AV-299, ARQ-197, MetMAb, CGEN-241, BMS-777607, JNJ-38877605, PF-4217903, SGX-126, CEP-17940, AMG-458, INCB-028060, and E-7050.

An example for a Notch pathway inhibitor is MEGF0444A.

An example for a c-Myc inhibitor is CX-3543.

Examples for Flt3 inhibitors are AC-220 (also an inhibitor of cKit and PDGFR), KW-2449, LS-104 (also an inhibitor of bcr-abl and Jak2), MC-2002, SB-1317, lestaurtinib (also an inhibitor of VEGFR, PDGFR, PKC), TG-101348 (also an inhibitor of JAK2), XL-999 (also an inhibitor of cKit, FGFR, PDGFR and VEGFR), sunitinib (also an inhibitor of PDGFR, VEGFR and cKit), and tandutinib (also an inhibitor of PDGFR, and cKit).

Examples for HSP90 inhibitors are, tanespimycin, alvespimycin, IPI-504, STA-9090, MEDI-561, AUY-922, CNF-2024, and SNX-5422.

Examples for JAK/STAT inhibitors are CYT-997 (also interacting with tubulin), TG-101348 (also an inhibitor of Flt3), and XL-019.

Examples for Mek inhibitors are ARRY-142886, AS-703026, PD-325901, AZD-8330, ARRY-704, RDEA-119, and XL-518.

Examples for mTor inhibitors are temsirolimus, deforolimus (which also acts as a VEGF inhibitor), everolimus (a VEGF inhibitor in addition), XL-765 (also a P13 kinase inhibitor), and BEZ-235 (also a P13 kinase inhibitor).

Examples for Akt inhibitors are perifosine, GSK-690693, RX-0201, and triciribine.

Examples for cKit inhibitors are masitinib, OSI-930 (also acts as a VEGFR inhibitor), AC-220 (also an inhibitor of Flt3 and PDGFR), tandutinib (also an inhibitor of Flt3 and PDGFR), axitinib (also an inhibitor of VEGFR and PDGFR), sunitinib (also an inhibitor of Flt3, PDGFR, VEGFR), and XL-820 (also acts as a VEGFR- and PDGFR inhibitor), imatinib (also a bcr-abl inhibitor), nilotinib (also an inhibitor of bcr-abl and PDGFR).

Examples for hedgehog antagonists are IPI-609, CUR-61414, GDC-0449, IPI-926, and XL-139.

Examples for CDK inhibitors are seliciclib, AT-7519, P-276, ZK-CDK (also inhibiting VEGFR2 and PDGFR), PD-332991, R-547, SNS-032, PHA-690509, PHA-848125, and SCH-727965.

Examples for proteasome inhibitors are bortezomib, carfilzomib, and NPI-0052 (also an inhibitor of NFkappaB).

Examples for proteasome inhibitors/NFkappaB pathway inhibitors are bortezomib, carfilzomib, NPI-0052, CEP-18770, MLN-2238, PR-047, PR-957, AVE-8680, and SPC-839.

An example for an inhibitor of the ubiquitination pathway is HBX-41108.

Examples for demethylating agends are 5-azacitidine and decitabine.

Examples for anti-angiogenic agents are inhibitors of the FGFR, PDGFR and VEGFR, and thalidomides, such agents being selected from, without limitation, olaratumab, pegdinetanib, motesanib, CDP-791, SU-14813, telatinib, KRN-951, ZK-CDK (also an inhibitor of CDK), ABT-869, BMS-690514, RAF-265, IMC-KDR, IMC-18F1, IMiDs, thalidomide, CC-4047, lenalidomide, ENMD-0995, IMC-D11, Ki-23057, brivanib, cediranib, 1B3, CP-868596, IMC-3G3, R-1530 (also an inhibitor of Flt3), sunitinib (also an inhibitor of cKit and Flt3), axitinib (also an inhibitor of cKit), lestaurtinib (also an inhibitor of Flt3 and PKC), vatalanib, tandutinib (also an inhibitor of Flt3 and cKit), pazopanib, PF-337210, E-7080, CHIR-258, sorafenib tosylate (also an inhibitor of Raf), vandetanib, CP-547632, OSI-930, AEE-788 (also an inhibitor of EGFR and Her2), BAY-57-9352 (also an inhibitor of Raf), BAY-73-4506 (also an inhibitor of Raf), XL-880 (also an inhibitor of cMet), XL-647 (also an inhibitor of EGFR and EphB4), XL-820 (also an inhibitor of cKit), nilotinib (also an inhibitor of cKit and brc-abl), CYT-116, PTC-299, BMS-584622, CEP-11981, dovitinib, CY-2401401, ENMD-2976, ramucirumab, pegdinetanib and BIBF1120.

The anti-neoplastic agent may also be selected from EGFR inhibitors, it may be a small molecule EGFR inhibitor or an anti-EGFR antibody. Examples for anti-EGFR antibodies, without limitation, are cetuximab, panitumumab, nimotuzumab, zalutumumab; examples for small molecule EGFR inhibitors are gefitinib, erlotinib, vandetanib (also an inhibitor of the VEGFR) and afatinib (also an inhibitor of Her2). Another example for an EGFR modulator is the EGF fusion toxin.

Further EGFR and/or Her2 inhibitors useful for combination with an Pharmaceutical combinations herein of the invention are lapatinib, trastuzumab, pertuzumab, XL-647, neratinib, BMS-599626 ARRY-334543, AV-412, mAB-806, BMS-690514, JNJ-26483327, AEE-788 (also an inhibitor of VEGFR), AZD-8931, ARRY-380 ARRY-333786, IMC-11F8, Zemab, TAK-285, AZD-4769, and afatinib (dual inhibitor of Her2 and EGFR).

DNA polymerase inhibitors useful in the combination with pharmaceutical combinations herein are Ara-C/cytarabine, Clolar/clofarabine.

A DNA methyltransferase inhibitor useful in the combination with pharmaceutical combinations herein is Vidaza/azacitidine.

An apoptosis inducer useful in the combination with pharmaceutical combinations herein is Trisenox/arsenice trioxide.

Topoisomerase II inhibitors useful in the combination with pharmaceutical combinations herein are idarubicin, daunorubicin and mitoxantrone.

A RAR antagonist useful in the combination with pharmaceutical combinations herein is Vesanoid/tretinoin.

A HGPRTase inhibitor useful in the combination with pharmaceutical combinations herein is Mercapto/mercaptopurine.

A histamine H2 receptor antagonist useful in the combination with pharmaceutical combinations herein is Ceplene/histamine dihydrochloride.

A CD25 receptor agonist useful in the combination with pharmaceutical combinations herein is IL-2.

The anti-neoplastic agent may also be selected from agents that target the IGF-1R and insulin receptor pathways. Such agents include antibodies that bind to IGF-1R (e.g. CP-751871, AMG-479, IMC-A12, MK-0646, AVE-1642, R-1507, BIIB-022, SCH-717454, rhu Mab IGFR) and novel chemical entities that target the kinase domain of the IGF1-R (e.g. OSI-906 or BMS-554417, XL-228, BMS-754807).

Other anti-neoplastic agents that may be advantageously combined in a therapy with the pharmaceutical combinations herein of the invention are molecules targeting CD20, including CD20 specific antibodies like rituximab, LY-2469298, ocrelizumab, MEDI-552, IMMU-106, GA-101 (=R7159), XmAb-0367, ofatumumab, radiolabeled CD20 antibodies, like tositumumab and ibritumomab tiuxetan or other CD20 directed proteins, like the SMIP Tru015, PRO-131921, FBT-A05, veltuzumab, R-7159.

Pharmaceutical combinations herein may be combined with inhibitors of other surface antigens expressed on leukocytes, in particular antibodies or antibody-like molecules, e.g. anti-CD2 (siplizumab), anti-CD4 (zanolimumab), anti-CD19 (MT-103, MDX-1342, SAR-3419, XmAb-5574), anti-CD22 (epratuzumab), anti-CD23 (lumiliximab), anti-CD30 (iratumumab), anti-CD32B (MGA-321), anti-CD38 (HuMax-CD38), anti-CD40 (SGN40), anti-CD52 (alemtuzumab), anti-CD80 (galiximab).

Other agents to be combined with pharmaceutical combinations herein are immunotoxins like BL-22 (an anti-CD22 immunotoxin), inotuzumab ozogamicin (an anti-CD23 antibody-calicheamicin conjugate), RFT5.dgA (anti-CD25 Ricin toxin A-chain), SGN-35 (an anti-CD30-auristatin E conjugate), and gemtuzumab ozogamicin (an anti-CD33 calicheamicin conjugate), MDX-1411 (anti-CD70 conjugate), or radiolabelled antibodies like ⁹⁰Y-epratuzumab (anti-CD22 radioimmunoconjugate).

In addition, pharmaceutical combinations herein may be combined with immunomodulators, agents, e.g. antibodies, that induce apoptosis or modify signal transduction pathways like the TRAIL receptor modulators mapatumumab (a TRAIL-1 receptor agonist), lexatumumab (a TRAIL-2 receptor agonist), tigatuzumab, Apomab, AMG-951 and AMG-655; an anti-HLA-DR antibody (like 1D09C3), an anti-CD74, an osteoclast differentiation factor ligand inhibitor (like denosumab), a BAFF antagonist (like AMG-623a) or an agonist of a Toll-like receptor (e.g. TLR-4 or TLR-9).

Other anti-neoplastic agents that may be used in combination with the pharmaceutical combinations herein of the present invention are selected from, but not limited to hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortinsone, fluoxymesterone, medroxyprogesterone, hydroxyprogesterone caproate, diethylstilbestrol, testosterone propionate, fluoxymesterone/equivalents, octreotide, arzoxifene, pasireotide, vapreotide, adrenocorticosteroids/antagonists, prednisone, dexamethasone, ainoglutethimide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, trimetrexate, pemetrexed, pyrimidine analogues like 5-fluorouracil, fluorodeoxyuridine, capecitabine, decitabine, nelarabine, 5-azacytidine, and gemcitabine, purine and adenosine analogues such as mercaptopurine, thioguanine, azathioprine, cladribine and pentostatin, cytarabine, fludarabine, clofarabine); antitumor antibiotics (e.g. anthracyclines like doxorubicin, daunorubicin, epirubicin and idarubicin, mitomycin-C, bleomycin dactinomycin, plicamycin, splicamycin, actimomycin D, mitoxantrone, mitoxantroneidarubicin, pixantrone, streptozocin, aphidicolin); platinum derivatives (e.g. cisplatin, oxaliplatin, carboplatin, lobaplatin, satraplatin); alkylating agents (e.g. estramustine, semustine, mechlorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan, banoxantrone, camptothecin) and miscellaneous chemotherapeutics such as retinoic acid derivatives, amifostine, anagrelide, interferon alpha, interferon beta, interferon gamma, interleukin-2, procarbazine, N-methylhydrazine, mitotane, and porfimer, bexarotene, celecoxib, ethylenemine/methyl-melamine, thriethylenemelamine, triethylene thiophosphoramide, hexamethylmelamine, and enzymes L-asparaginase, L-arginase and metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine, erythrohydroxynonyl-adenine, anthracenedione, GRN-163L (a competitive telomerase template antagonist), SDX-101 (a PPAR agonist), talabostat (a DPP inhibitor), forodesine (a PNP inhibitor), atacicept (a soluble receptor targeting TNF family members BLyS and APRIL), TNF-alpha neutralizing agents (Enbrel, Humira, Remicade), XL-844 (a CHK1/2 inhibitor), VNP-40101M (a DNA alkylating agent), SPC-2996 (an antisense bcl2 inhibitor), obatoclax (a bcl2 inhibitor), enzastaurin (a PKC beta modulator), vorinistat (an HDAC inhibitor), romidepsin (an HDAC inhibitor), AT-101 (a Bcl-2/Bcl-xL inhibitor), plitidepsin (a multi-actioned depsipeptide), SL-11047 (a polyamine metabolism modulators).

The pharmaceutical combinations herein of the invention may also be used in combination with other therapies including surgery, stem cell transplantation, radiotherapy, endocrine therapy, biologic response modifiers, hyperthermia and cryotherapy and agents to attenuate any adverse effect (e.g. antiemetics), G-CSF, GM-CSF, photosensitizers such as hematoporphyrin derivatives, Photofrin, benzoporphyrin derivatives, Npe6, tin etioporphyrin, pheoboride-a bacteriochlorophyll-a, naphthalocyanines, phthalocyanines, zinc phthalocyanines.

### Pharmaceutical Compositions and Methods of Administration

“Pharmaceutical composition” as used herein refers to a means to make the pharmaceutical combinations herein administrable to a patient. This means that the pharmaceutical combination as active ingredients of the pharmaceutical composition is admixed with one or more pharmaceutically acceptable diluents and optionally further pharmaceutically acceptable agents. The pharmaceutical composition herein can be in any form that allows for the pharmaceutical composition to be administered to a patient. For example, the pharmaceutical composition can be in the form of a solid or liquid. The preferred mode of application is parenteral, by infusion or injection (intravenous, intramuscular, subcutaneous, intraperitoneal, intradermal), but other modes of application such as by inhalation, transdermal, intranasal, buccal, oral and intra-tumor may also be applicable. Parenteral administration includes subcutaneous injections, intravenous, intramuscular, intrastemal injection or infusion techniques. In one aspect, the pharmaceutical compositions are administered parenterally. In yet another aspect, the pharmaceutical compositions are administered intravenously.

Pharmaceutical compositions can be formulated so as to allow a compound to be bioavailable upon administration of the pharmaceutical composition to a patient. Pharmaceutical compositions can take the form of one or more dosage units, where, for example, a container of a compound in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of patient (e.g., human), the particular form of the active constituents (i.e. dual anti-Ang2/anti-Dll4 binders and anti-VEGF agents, optionally anti-neoplastic agents), the manner of administration, and the pharmaceutical composition employed.

The pharmaceutically acceptable carrier or vehicle can be particulate, so that the pharmaceutical compositions are, for example, in powder form. The carrier(s) can be liquid, with the pharmaceutical compositions being, for example, an injectable liquid. The pharmaceutical composition can be in the form of a liquid, e.g., for parenteral injection. In a pharmaceutical composition for administration by injection, one or more of a surfactant, preservative, wetting agent, dispersing agent, suspending agent, buffer, stabilizer and isotonic agent can also be included.

The liquid pharmaceutical compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, cyclodextrin, propylene glycol or other solvents; stabilizers such as amino acids; surfactants such as polysorbates; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. A parenteral pharmaceutical composition can be enclosed in ampoule, a disposable syringe or a multiple-dose vial made of glass, plastic or other material. Physiological saline is an exemplary adjuvant. An injectable pharmaceutical composition is preferably sterile.

The pharmaceutical compositions herein may also be dried (freeze-dried, spray-dried, spray-freeze dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, nano-particles and nanocapsules), in macroemulsions or precipitated or immobilized onto carriers or surfaces, for example by pcmc technology (protein coated microcrystals). Such techniques are disclosed in Remington: The Science and Practice of Pharmacy, 21st edition, Hendrickson R. Ed.

An example for an anti-VEGF agent is Bevacizumab, which is marketed in many countries under the trade name “Avastin®”. Avastin® can be used for the purposes of the present invention in any formulation for intravenous or intra-arterial application.

The dual anti-Ang2/anti-Dll4 binder is typically formulated as infusion solution for intravenous application. As a typical example BI-1 can be formulated as follows:

| | | |
|---|---|---|
| BI-1 | 0.492 | mmol/l |
| Disodium succinate hexahydrate | 22.3 | mmol/l |
| Succinic acid | 2.7 | mmol/l |
| Trehalose dehydrate | 155.0 | mmol/l |
| 2-Hydroxypropyl-β-Cyclodextrin | 32.436 | mmol/l |
| Polysorbate 20 (Tween 20) | 0.244 | mmol/l |
| Water for injection (WFI) | ad 1 | liter |

Also other suitable infusions solution formulations known in the art can be used.

The amount of the pharmaceutical composition that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the pharmaceutical compositions will also depend on the route of administration, and the seriousness of the disease or disorder, and should be decided according to the judgment of the practitioner and each patient's circumstances.

The pharmaceutical compositions comprise an effective amount of a drug(s) or agent(s) such that a suitable dosage will be obtained. Typically, this amount is at least about 0.01% of a drug or agent by weight of the pharmaceutical composition. When intended for oral administration, this amount can be varied to range from about 0.1% to about 80% by weight of the pharmaceutical composition. In one aspect, oral pharmaceutical compositions can comprise from about 4% to about 50% of the active constituents by weight of the pharmaceutical composition. In yet another aspect, present pharmaceutical compositions are prepared so that a parenteral dosage unit contains from about 0.01% to about 2% by weight of the active constituents.

For intravenous administration, the pharmaceutical composition can comprise from about 1 to about 50 mg of a drug or agent per kg of the patient's body weight. In one aspect, the pharmaceutical composition can include from about 1, 1.5 or 2.5 to about 50 mg of a drug or agent per kg of the patient's body weight. In another aspect, the amount administered will be in the range from about 1, 1.5 or 2.5 to about 25 mg/kg of body weight of a drug or agent.

In some embodiments, the dosage administered to a patient is less than 0.1 mg/kg to about 50 mg/kg of the patient's body weight. (For conversion to mg/mm2, a BSA of 1.8 m2 and a body weight of 80 kg can be used.)

As discussed herein, pharmaceutical compositions herein can be administered intravenously or subcutaneously to the patient on a schedule that is, for example, daily, weekly, biweekly, tri-weekly or monthly to the patient. For example, pharmaceutical compositions herein can be administered weekly, for a period of 2 to 10 weeks, typically 3-6 weeks. In some embodiments, the dosage regimen of the pharmaceutical compositions herein maintains a blood serum concentration of antibody at least 5 μg/ml or at least 10 μg/ml during the dosage cycle. The pharmaceutical compositions herein can be administered, for example, from 1-8, or more cycles. In some embodiments, pharmaceutical compositions herein are administered chronically to a subject.

By way of example, the invention includes a method of treating a cancer, such as myeloid leukemia, by administering 0.1 mg/kg to 50 mg/kg, for instance about 1.5-8 or 2.5-8 mg/kg, of a pharmaceutical composition herein weekly. This treatment can be usually continued for about 1-3 months, typically about two months. In an embodiment, the dosing schedule is maintained until a reduction in blasts is noted. For example, dosing can be continued up to about 6 months. This treatment can be followed by a less frequent dosing schedule, involving for instance biweekly doses (or twice per month). This dosing schedule can be maintained 1, 2, 3, 4, 5, 6 months or more to maintain a reduction in blasts and/or a remission.

In some embodiments, a prophylactic agent can be administered with pharmaceutical compositions herein to minimize infusion reactions. Suitable prophylactic agents include, for example, methyl prednisolone, diphenyldramine, acetaminophen or other suitable agent. The prophylactic agent can be administered prior to or at about the same time as the pharmaceutical compositions herein.

The pharmaceutical compositions herein can be administered by any convenient route, for example, by infusion or bolus injection, by absorption through epithelial or mucocutaneous linings (e.g., oral mucosa, rectal and intestinal mucosa, etc.). Administration can be systemic or local. Various delivery systems are known, e.g., encapsulation in liposomes, microparticles, microcapsules, capsules, etc., and can be used to administer the pharmaceutical compositions herein.

It can be desirable to administer the pharmaceutical compositions herein locally to the area in need of treatment, as appropriate for the drug or agent. This can be achieved, for example, and not by way of limitation, by local infusion during surgery; topical application, e.g., in conjunction with a wound dressing after surgery; by injection: by means of a catheter; by means of a suppository; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue.

The pharmaceutical compositions herein can be delivered in a controlled release system, such as a pump or various polymeric materials. In yet another embodiment, a controlled-release system can be placed in proximity of the target of the pharmaceutical compositions herein, thus requiring only a fraction of the systemic dose (see, e.g., Goodson, in Medical Applications of Controlled Release, vol. 2, pp. 115-138, 1984). Other controlled-release systems discussed in the review by Langer (1990, Science 249: 1527-1533) can be used.

The pharmaceutical compositions herein are formulated in accordance with routine procedures as a pharmaceutical composition adapted for intravenous administration to animals, particularly human beings, as appropriate for the drug or agent. Typically, the carriers or vehicles for intravenous administration are sterile isotonic aqueous buffer solutions. Where necessary, the pharmaceutical compositions can also include a solubilizing agent. Pharmaceutical compositions for intravenous administration can optionally comprise a local anesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where drug or agent is to be administered by infusion, it can be dispensed, for example, with an infusion bottle containing sterile pharmaceutical grade water or saline. Where the drug or agent is administered by injection, an ampoule of sterile water for injection or saline can be provided so that the ingredients can be mixed prior to administration.

Pharmaceutical compositions of therapeutic agents also can be administered according to accepted dosage forms in the form of tablets, lozenges, aqueous or oily suspensions, granules, powders, emulsions, capsules, syrups, or elixirs, for example. Orally administered pharmaceutical compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the pharmaceutical compositions can be coated to delay disintegration and absorption in the gastrointestinal tract thereby providing a sustained action over an extended period of time. Selectively permeable membranes surrounding an osmotically active driving compound are also suitable for orally administered drugs or agents. In these later platforms, fluid from the environment surrounding the capsule is imbibed by the driving compound, which swells to displace the agent or agent pharmaceutical composition through an aperture. These delivery platforms can provide an essentially zero order delivery profile as opposed to the spiked profiles of immediate release formulations. A time-delay material such as glycerol monostearate or glycerol stearate can also be used.

The pharmaceutical composition can include various materials that modify the physical form of a solid or liquid dosage unit. For example, the pharmaceutical composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The pharmaceutical compositions can be administered to a patient in need thereof at a frequency, or over a period of time, that is determined by the attending physician. The pharmaceutical compositions can be administered over a period of 1 day, 2 days, 3 days, 5 days, 7 days, 10 days, 14 days, 21 days, 28 days, one month, two months, or longer periods of time. It is understood that the pharmaceutical compositions can be administered for any period of time between 1 day and two months or longer.

The combinations may be presented as a combined preparation kit. By the term “combined preparation kit” or “kit” as used herein is meant the pharmaceutical composition or compositions that are used to administer the pharmaceutical combinations according to the invention. When the active constituents of the pharmaceutical combinations, i.e. the anti-Ang2/anti-Dll4 binders and anti-VEGF agents and optionally the anti-neoplastic agent(s) are administered simultaneously, the combined preparation kit can contain each active constituent in a single pharmaceutical composition, such as a tablet, or in separate pharmaceutical compositions. When the active constituents are not administered simultaneously, the combined preparation kit will contain the active constituents in separate pharmaceutical compositions either in a single package or the active constituents in separate pharmaceutical compositions in separate packages or compartments.

In one aspect there is provided a pharmaceutical composition in the form of a combined preparation kit comprising

(i) a first compartment containing a first pharmaceutical composition comprising a the anti-Ang2/anti-Dll4 binder;
(ii) a second compartment containing a second pharmaceutical composition comprising anti-VEGF agent; and optionally
(iii) a third compartment containing one or more pharmaceutical composition(s) comprising one or more additional anti-neoplastic agent(s).

In one embodiment there is provided a combined preparation kit comprising the active constituents as suitable pharmaceutical compositions, wherein the active constituents are provided in a form which is suitable for sequential, separate and/or simultaneous administration.

In one embodiment there is provided a combined preparation kit comprising the following components: a first container comprising a anti-Ang2/anti-Dll4 binder as a suitable pharmaceutical composition; and a second container comprising an anti-VEGF agent as a suitable pharmaceutical composition, and a container means for containing said first and second containers.

The combination kit can also be provided by instruction, such as dosage and administration instructions. Such dosage and administration instructions can be of the kind that are provided to a doctor, for example by a drug product label, or they can be of the kind that are provided by a doctor, such as instructions to a patient.

In another aspect, the present invention also relates to dual anti-Ang2/anti-Dll4 binders for use in the treatment of cancer in combination with anti-VEGF agents.

In another aspect, the present invention relates to a method of treatment of cancer, comprising administration of a therapeutically effective amount of a dual anti-Ang2/anti-Dll4 binder to a patient in need thereof, and furthermore comprising administration of a therapeutically effective amount of an anti-VEGF agent to the same patient within 72 hours before or after administration of said dual anti-Ang2/anti-Dll4 binder.

In another embodiment the administration of the anti-VEGF agent is done within 36 hours before or after administration of said dual anti-Ang2/anti-Dll4 binder.

In another embodiment the administration of the anti-VEGF agent is done within 24 hours before or after administration of said dual anti-Ang2/anti-Dll4 binder.

In another embodiment the administration of the anti-VEGF agent is done within 12 hours before or after administration of said dual anti-Ang2/anti-Dll4 binder.

In another embodiment the administration of the anti-VEGF agent is done within 6 hours before or after administration of said dual anti-Ang2/anti-Dll4 binder.

In another embodiment the administration of the anti-VEGF agent is done within 3 hours before or after administration of said dual anti-Ang2/anti-Dll4 binder.

In another embodiment the administration of the anti-VEGF agent is done within 2 hours before or after administration of said dual anti-Ang2/anti-Dll4 binder.

In another embodiment the administration of the anti-VEGF agent is done within 1 hours before or after administration of said dual anti-Ang2/anti-Dll4 binder.

In another embodiment the administration of the anti-VEGF agent is done within 30 minutes before or after administration of said dual anti-Ang2/anti-Dll4 binder.

In another embodiment the administration of the anti-VEGF agent is done simultaneously with the administration of said dual anti-Ang2/anti-Dll4 binder.

Simultaneous administration of the anti-VEGF agent and the dual anti-Ang2/anti-Dll4 binder can typically be achieved by
Administering both anti-VEGF agent and dual anti-Ang2/anti-Dll4 binder by simultaneous infusion out of separate infusion vessels, or by
Administering both anti-VEGF agent and dual anti-Ang2/anti-Dll4 binder by simultaneous infusion out of the same infusion vessel, or by
Administering anti-VEGF agent subcutaneously while administering the dual anti-Ang2/anti-Dll4 binder by infusion, or by
Administering anti-VEGF agent by infusion while administering the dual anti-Ang2/anti-Dll4 binder subcutaneously, or by
Administering both anti-VEGF agent and the dual anti-Ang2/anti-Dll4 binder subcutaneously.

### EXPERIMENTAL PART

### Acronyms and Abbreviations

### FCS Fetal Calf Serum

h hour

### IgG Immunoglobulin G

### PBS Phosphate-Buffered Saline

TGI Tumor Growth Inhibition, calculated to the formula:

TGI=100×{1−[(treatedfinal day−treatedday1)/(controlfinal day−controlday1)]}

1. In Vivo Efficacy of BI-1 in Combination with Bevacizumab and BIBF 1120 in a Mouse Model of Human Non-Small Cell Lung Cancer (NCl-H1975)

The goal of the present study was to assess the efficacy of BI-1 in combination with Bevacizumab and BIBF 1120 in a model of human non small cell lung cancer (NCl-H1975) in nude mice.

### 1.1 Materials and Methods

### 1.1.1 Study Design

Model: Subcutaneous xenografts of the human non-small cell lung cancer (NCl-H1975) growing in nude mice

| | | | | Schedule [days | |
|---|---|---|---|---|---|
| | Number | | Dose | of admin. per | |
| Group | of mice | Compound | [mg/kg] | week] | Route |
| 1 | 10 | Vehicle control | — | q3or4d | i.p. |
| | | (NaCl 0.9%) | | | |
| 2 | 7 | Bevacizumab | 25 | q3or4d | i.p. |
| 3 | 7 | BIBF 1120 | 50 | qdx7 | p.o. |
| 4 | 7 | BI-1 | 13.6 | q3or4d | i.p. |
| 5 | 7 | Bevacizumab + | 25 + | q3or4d + | i.p. + |
| | | BI-1 | 13.6 | q3or4d | i.p. |
| 6 | 7 | BIBF 1120 + | 50 + | qd + | p.o. + |
| | | BI-1 | 13.6 | q3or4d | i.p. |

### 1.1.2 Test Compounds

B1-1 with the sample ID number D11B20V503 was used for this experiment and diluted with PBS. BIBF 1120 with the batch chiffre 133562 was suspended in Natrosol 0.5% (Hydroxyethylcellulose Natrosol 250 HX, VWR). Avastin® (Bevacizumab, 25 mg/ml) was purchased from Roche (Basel, Switzerland), (dissolved in 0.9% saline) was diluted with 0.9% saline.

### 1.1.3 Mice

Mice were 7 week-old female BomTac:NMRI-Foxn1nu purchased from Taconic, Denmark. After arrival, mice were allowed to adjust to ambient conditions for at least 5 days before they were used for the experiments. They were housed in Makrolon® type III cages in groups of 7 (10 for the controls) under standardized conditions at 21.5±1.5° C. temperature and 55±10% humidity. Standardized diet (PROVIMI KLIBA) and autoclaved tap water were provided ad libitum. Subcutaneously implanted (under isoflurane anesthesia) microchips were used to identify each mouse. Cage cards showing the study number, the animal identification number, the compound and dose level, the administration route as well as the schedule remained with the animals throughout the study.

### 1.1.4 Establishment of Tumors, Randomization

To establish subcutaneous tumors, NCl-H1975 cells were harvested by centrifugation, washed and resuspended in PBS+5% FCS at 5×107 cells/ml. 100 μl cell suspension containing 5×106 cells was then injected subcutaneously into the right flank of the mice (1 site per mouse). Mice were randomly distributed between the treatment and the vehicle control group (7 days after cell injection) when tumors were well established and had reached volumes of 63 to 104 mm3.

### 1.1.5 Administration of Test Compound

The doses of BI-1 and Bevacizumab were calculated to the average body weight of all mice on day 1 (28 g) and administered intraperitoneally twice weekly in a volume of 100 μl per mouse. BIBF 1120 was dosed according to the body weight (mg/kg) and administered daily perorally.

### 1.1.6 Monitoring Tumor Growth and Side Effects

Tumor diameters were measured three times a week (Monday, Wednesday and Friday) with a caliper. The volume of each tumor [in mm3] was calculated according to the formula “tumor volume=length*diameter2*π/6.” To monitor side effects of treatment, mice were inspected daily for abnormalities and body weight was determined three times a week (Monday, Wednesday and Friday). Animals were sacrificed when the control tumors reached a size of approximately 800 mm3 on average. In addition, animals with tumor sizes exceeding 1.5 cm in diameter or 20% body weight loss were euthanized for ethical reasons.

TGI values were calculated as follows:

TGI=100×{1−[(treated final day−treated day1)/(control final day−control day1)]}

### 1.1.7 Tumor Sampling

At euthanasia (24 h after the last oral and 4 days after the last intraperitoneal treatment, respectively) five tumors per group were excised and placed into cryo tubes to be snap frozen in liquid nitrogen and stored at −80° C.

### 1.1.8 Statistical Analysis

The statistical evaluation was performed for the parameters tumor volume and body weight at day 14.

For the tumor volume absolute values and for the body weight the percentage change referred to the initial weight of day 1 was used.

Due to the observed variation nonparametric methods were applied.

For descriptive considerations the number of observations and the median were calculated. For a quick overview of possible treatment effects the median of the tumor volume of each treatment group T was referred to the median of the control C as

Tumor growth inhibition (TGI) from day 1 until day d

TGI=100*[(Cd−C1)−(Td−T1)]/(Cd−C1)
where C1, T1=median tumor volumes in control and treatment group
   at start of the experiment at day 1,
Cd, Td=median tumor volumes in control and treatment group
   at day 14

One-sided decreasing Mann-Whitney tests were applied to compare each treatment group with the control, as well as the mono therapies with the corresponding combination therapy, looking for a reduction in tumor volume as effect and a reduction in the body weight gain as adverse event.

The p values for the tumor volume were adjusted for multiple comparisons according to Bonferroni-Holm within each subtopic (comparisons versus control, comparisons combination versus single agent therapy) whereas the p values of the body weight (tolerability parameter) remained unadjusted in order not to overlook a possible adverse effect.

The level of significance was fixed at α=5%. An (adjusted) p value of less than 0.05 was considered to show a statistically significant difference between the groups and differences were seen as indicative whenever 0.05≦p value<0.10.

### 1.2 Results

### 1.2.1 Tumor Volume—Single Agents

During the 14 day treatment period, control tumors grew from a median volume of 85 mm³ to a volume of 791 mm³.

Treatment with 25 mg/kg Bevacizumab administered twice weekly i.p. for 2.5 cycles significantly delayed tumor growth (median TGI=82%, p=0.0010).

Treatment with 50 mg/kg BIBF 1120 administered daily p.o. for 2.5 cycles significantly delayed tumor growth (median TGI=75%, p=0.0010).

Treatment with 13.6 mg/kg BI-1 administered twice weekly i.p. for 2.5 cycles significantly delayed tumor growth (median TGI=75%, p=0.0010).

Treatment with 25 mg/kg Bevacizumab and 13.6 mg/kg BI-1 administered twice weekly i.p. for 2.5 cycles significantly delayed tumors growth (median TGI=99%, p=0.0010).

Treatment with 50 mg/kg BIBF 1120 administered daily p.o. and 13.6 mg/kg BI-1 administered twice weekly i.p. for 2.5 cycles significantly delayed tumors growth (median TGI=98%, p=0.0010).

### 1.2.2 Tumor Volume—Combinations

The combination of Bevacizumab and BI-1 was significantly more effective than Bevacizumab (p=0.0012) or BI-1 (p=0.0006) alone.

The combination of BIBF 1120 and BI-1 was significantly more effective than BIBF 1120 (p=0.0006) or BI-1 (p=0.0006) alone.

### 1.2.3 Body Weight

The control animals gained 6.0% body weight. The body weight gain of all treatment groups was comparable to the controls (no significant differences).

### 1.3 Conclusion

Bevacizumab, BIBF 1120, BI-1, the combination of Bevacizumab with BI-1 and the combination of BIBF 1120 with BI-1 all significantly delayed NCl-H1975 tumor growth.

The combinations of Bevacizumab with BI-1 and BIBF 1120 with BI-1 were both significantly more effective than the corresponding single agents. All therapies were well tolerated.

Based on the findings gained from the experiment described above it can be concluded that pharmaceutical combinations comprising a dual anti-Ang2/anti-Dll4 binders and an anti-VEGF agents indeed have a superior anti-angiogenic efficacy and thus, as presented, also a superior anti-cancer efficacy. It has also been shown that such pharmaceutical combinations are well tolerable for the patients since there was no decrease in body weight with all animals over the duration of the experiment.

2. In Vivo Efficacy of BI-1 in Combination with Bevacizumab and BIBF1120 in Mouse Models of Human Non-Small Cell Lung Cancer

The goal of the present study was to assess the efficacy of BI-1 in combination with Bevacizumab, BIBF1120 or Sunitinib in models of human non small cell lung cancer (LXFE 211, LXFE 1422), colon cancer (CXF 243), mammary cancer (MAXF 401), ovarian cancer (OVXF 1353), pancreatic cancer (PAXF 546) and renal cancer (RXF 1220) in nude mice. All models were patient-derived tumor xenografts (PDX), which were transplanted from patients to nude mice and passaged subcutaneously. These models retain most of the characteristics of the parental patient tumors including histology.

### 2.1 Materials and Methods

### 2.1.1 Study Design

Model: LXFE 211, LXFE 1422, CXF 243, MAXF 401, OVXF 1353 and PAXF 546

| | | | | | No. |
|---|---|---|---|---|---|
| | | Dose | Schedule | | of |
| Group | Treatment | [mg/kg/dose] | [day] | Route | mice |
| 1 | Vehicle | 100 μl/mouse | Twice weekly | i.p. | 10 |
| 2 | Bevacizumab | 15 | Twice weekly | i.p. | 10 |
| 3 | BI-1 | 13.6 | Twice weekly | i.p. | 10 |
| 4 | BIBF1120 | 50 | Once daily | p.o. | 10 |
| 5 | BI-1 + | 13.6 | Twice weekly | i.p. | 10 |
| | Bevacizumab | 15 | | i.p. | |
| 6 | BI-1 + | 13.6 | Twice weekly | i.p. | 10 |
| | BIBF1120 | 50 | Once daily | p.o. | |

Model: RXF 1220

| | | | | | No. |
|---|---|---|---|---|---|
| | | Dose | Schedule | | of |
| Group | Treatment | [mg/kg/dose] | [day] | Route | mice |
| 1 | Vehicle | 100 μl/mouse | Twice weekly | i.p. | 10 |
| 2 | Bevacizumab | 15 | Twice weekly | i.p. | 10 |
| 3 | BI-1 | 13.6 | Twice weekly | i.p. | 10 |
| 4 | Sunitinib | 40 | Once daily | i.p. | 10 |
| 5 | BI-1 + | 13.6 | Twice weekly | i.p. | 10 |
| | Bevacizumab | 15 | Twice weekly | i.p. | |
| 6 | BI-1 + | 13.6 | Twice weekly | i.p. | 10 |
| | Sunitinib | 40 | Once daily | p.o. | |

### 2.1.2 Test Compounds

BI-1 with the sample ID number D11B20V503 was used for this experiment and diluted with PBS. BIBF1120 with the batch chiffre 133562 was suspended in Natrosol 0.5% (Hydroxyethylcellulose Natrosol 250 HX, VWR). Bevacizumab (Avastin®, 25 mg/ml) was purchased from Roche (Basel, Switzerland), dissolved in 0.9% saline, was diluted with 0.9% saline.

Sunitinib (Sutent®, Pfizer) tablets were ground with mortar and pestle and 108.48 mg powder (corresponding to 32 mg API; correction factor: 3.39) were dissolved in PBS (pH 5).

### 2.1.3 Mice

Mice were 5-7 week-old female Crl:NMRI-Foxn1^{nu} purchased from Charles River, Sulzfeld, Germany. After arrival, mice were allowed to adjust to ambient conditions for at least 5 days before they were used for the experiments. They were housed in individual ventilated Makrolon® type II long cages under standardized conditions at 25±1° C. temperatures and 55±10% humidity. Standardized diet (Teklad Global 19% Protein Extruded Diet (T.20195.12) from Harlan Laboratories) and sterile filtrated and acidified (pH 2.5) tap water were provided ad libitum. Ear clips were used to identify each mouse. Cage cards showing the study number, the animal identification number, the compound and dose level, the administration route as well as the schedule remained with the animals throughout the study.

### 2.1.4 Establishment of Tumors, Randomization

Tumor fragments were obtained from tumor xenografts in serial passage in nude mice. After removal from donor mice, tumors were cut into fragments (4-5 mm diameter) and placed in PBS until subcutaneous implantation. Recipient mice were anesthetized by inhalation of isoflurane. A small incision was made and one tumor fragment per animal was transplanted with tweezers. Mice were monitored daily.

At randomization, tumor-bearing animals were stratified into the various groups according to tumor volume. Only animals carrying a tumor of appropriate size (50-250 mm³ volume) were considered for randomization. Mice were randomized when the required number of mice qualified for randomization. The day of randomization was designated as day 0. The first day of dosing was day 1.

### 2.1.5 Administration of Test Compound

The doses of BI-1 and Bevacizumab were calculated to the average body weight of all mice on day 1 (28 g) and administered intraperitoneally twice weekly in a volume of 100 μl per mouse. BIBF1120 and Sunitinib were dosed according to the body weight (mg/kg) and administered daily perorally.

### 2.1.6 Monitoring Tumor Growth and Side Effects

Tumor diameters were measured twice weekly with a caliper. The volume of each tumor [in mm³] was calculated according to the formula “tumor volume=length*diameter²*0.5.” To monitor side effects of treatment, mice were inspected daily for abnormalities and body weight was determined twice weekly. Animals with tumor sizes exceeding 1.5 cm in diameter or 20% body weight loss were euthanized for ethical reasons.

TGI values were calculated as follows:

TGI=100×{1−[(treated final day−treated day1)/(control final day−control day1)]}

### 2.1.7 Tumor Sampling

At euthanasia (24 h after the last treatment) five tumors per group were excised and placed into cryo tubes to be snap frozen in liquid nitrogen and stored at −80° C.

### 2.1.8 Statistical Analysis

For the evaluation of the statistical significance of tumor inhibition a one-tailed non-parametric Mann-Whitney-Wilcoxon U-test was performed, based on the hypothesis that an effect would only be measurable in one direction (i.e. expectation of tumor inhibition but not tumor stimulation). In general, the U-test compares the ranking of the individual tumors of two groups, according to absolute volume on a particular day (pairwise comparisons between groups). Here it was used to compare the groups receiving combination therapy with the groups given the respective monotherapies. The p-values obtained from the U-test were adjusted using the Bonferroni-Holm correction. By convention, p-values≦50.05 indicate significance of differences.

### 2.2 Results

### 2.2.1 Tumor Volume

### BI-1/Bevacizumab Combination Therapy Versus BI-1 and Bevacizumab Monotherapies

BI-1/bevacizumab combination therapy displayed significant efficacy in all seven tumor xenografts with TGI values ranging from 84% for RXF 1220 to 106% for PAXF 546. The combination therapy was significantly more efficacious than the bevacizumab monotherapy in all seven tumor models (TGI values for bevacizumab between 10%-68%). The combination therapy was significantly more efficacious than the BI-1 monotherapy in LXFE 211, LXFE 1422, MAXF 401 and PAXF 546 (TGI values for BI-1 between 76% and 94%).

### BI-1/BIBF1120 Combination Therapy Versus BI-1 and BIBF1120 Monotherapies

BI-1/BIBF1120 combination therapy exhibited the strongest efficacy among the tested treatments in all six tumor xenografts in which it was tested (CXF 243, LXFE 211, LXFE 1422, MAXF 401, OVXF 1353, PAXF 546) with TGI values ranging from 95% with CXF 243 to 110% with MAXF 401. In all tested tumor models, the efficacy advantage over the corresponding monotherapies (range of TGI values for BI-01: 76% to 94%, for BI-20: 40% to 78%) was significant.

### BI-1/Sunitinib Combination Therapy Versus BI-1 and Sunitinib Monotherapies

Since sunitinib is registered for the treatment of metastatic renal cell cancer, the efficacy of BI-1/sunitinib combination therapy was only tested in mice bearing the RXF 1220 tumor xenograft. This treatment resulted in the TGI value of 103%. The efficacy advantages over the reference monotherapies with BI-1 (TGI value 76%) and sunitinib (62%) were significant.

### Summary of Results

| | | | | | | Pᵥₐₗᵤₑ vs. |
|---|---|---|---|---|---|---|
| | TGI [%] | Combination | TGI | TGI [%] | Pᵥₐₗᵤₑ vs. | combo |
| Model | BI-1 | partner | [%] | combination | BI-1 | partner |
| CXF 243 | 76 | BIBF1120 | 65 | 95 | 0.0434 | 0.0434 |
| LXFE 211 | 92 | Bevacizumab | 37 | 95 | 0.0394 | 0.0002 |
| LXFE 211 | 92 | BIBF1120 | 40 | 102 | 0.0012 | 0.0002 |
| LXFE 1422 | 94 | Bevacizumab | 63 | 99 | 0.0144 | 0.0002 |
| LXFE 1422 | 94 | BIBF1120 | 57 | 101 | 0.0002 | 0.0113 |
| MAXF 401 | 87 | Bevacizumab | 68 | 103 | 0.0446 | 0.0016 |
| MAXF 401 | 87 | BIBF1120 | 63 | 110 | 0.0093 | 0.0082 |
| OVXF 1353 | 88 | BIBF1120 | 78 | 102 | 0.0028 | 0.0007 |
| PAXF 546 | 94 | Bevacizumab | 59 | 106 | 0.0144 | 0.0022 |
| PAXF 546 | 94 | BIBF1120 | 69 | 107 | 0.0474 | 0.0003 |
| RXF 1220 | 76 | Sunitinib | 62 | 103 | 0.0008 | 0.0002 |

### 2.2.2 Body Weight

For all treatments, maximum group median body weight losses observed during experiments were generally below 5% and were usually comparable to those observed for the respective vehicle control groups. However, the following exceptions were recorded: (i) In the experiments with the cachexia-inducing tumor xenografts LXFE 211 and RXF 1220 for vehicle control groups maximum group median body weight losses of 5.8% and 13.7%, respectively, were observed. Moreover, in the experiment with LXFE 211, maximum median body weight losses of 9.1% and of 5.9%, respectively, were observed for the bevacizumab- and BI-20-treated groups, i.e. for the two treatments exhibiting the weakest anti-tumor efficacy. (ii) In the experiments with CXF 243 (maximum group median body weight loss: 10.2%), LXFE 1422 (3.4%), MAXF 401 (6.2%), OVXF 1353 (9.8%) and PAXF 546 (4.3%) the highest group median body weight losses were recorded for the group given the BI-1/BIBF1120 combination therapy. In addition, in the experiment with RXF 1220 the second highest maximum median body weight loss (4.5%) was recorded for the group dosed with the BI-1/sunitinib combination.

There was a trend towards a higher incidence of deaths in the groups that received either BI-01/BIBF1120 or bevacizumab/BI-01 combination therapy with 11 and six deaths over all experiments, respectively. These deaths occurred only after prolonged treatment (no death prior to exp. day 25). Separately, in the experiment with RXF 1220, 11 animals were euthanized due to body weight losses or were found dead. Since in this latter experiment most of the deaths occurred in the vehicle control group and in the bevacizumab-treated group, i.e. under the treatments with the weakest anti-tumor efficacy, it is likely that those deaths are related to tumor-induced cachexia. One reason for the higher number of deaths in the experiments with CXF 243 and OVXF 1353 (nine and six deaths, respectively) as compared to the other experiments is the long duration of both experiments (>8 and >7 weeks, respectively, for most of the groups).

### 2.3 Conclusion

BI-1 in monotherapy as well as BI-1/bevacizumab, BI-1/BIBF1120 and BI-1/sunitinib in combination therapy displayed significant anti-tumor efficacy in all seven tested tumor xenografts.

The tested combination therapies were in all cases significantly more efficacious than the respective monotherapies.

The combination of BI-1 with an NCE (either BIBF1120 or sunitinib) was a very efficacious treatment in all experiments (TGI: 95%-110%). Also the BI-1/bevacizumab combination (TGI: 84%-106%) yielded in high treatment efficacy.

Based on the findings gained from the experiment described above it can be concluded that pharmaceutical combinations comprising a dual anti-Ang2/anti-Dll4 binders and an anti-VEGF-R agents indeed have a superior anti-angiogenic efficacy and thus, as presented, also a superior anti-cancer efficacy. It has also been shown that such pharmaceutical combinations are well tolerable for the patients since there was no decrease in body weight with all animals over the duration of the experiment.

1. Pharmaceutical combinations comprising one or more dual anti-Ang2/anti-Dll4 binders and one or more anti-VEGF agents.

2. Pharmaceutical combinations according to claim 1, wherein the dual anti-Ang2/anti-Dll4 binders are selected from SeqID No: 1-20.

3. Pharmaceutical combinations according to claim 1, wherein the anti-VEGF agents are selected from bevacizumab, pegaptanib, ranibizumab, aflibercept and PRS-050.

4. Pharmaceutical combinations according to claim 1, further comprising one or more anti-neoplastic agents.

5. Pharmaceutical combinations according to claim 3, comprising a dual anti-Ang2/anti-Dll4 binder according to SeqID No: 14 and bevacizumab.

6. Pharmaceutical combinations according to claim 3, comprising a dual anti-Ang2/anti-Dll4 binder according to SeqID No: 14 and aflibercept.

7. Pharmaceutical combinations according to claim 3, comprising a dual anti-Ang2/anti-Dll4 binder according to SeqID No: 15 and bevacizumab.

8. Pharmaceutical combinations according to claim 3, comprising a dual anti-Ang2/anti-Dll4 binder according to SeqID No: 16 and bevacizumab.

9. Pharmaceutical combinations according to claim 3, comprising a dual anti-Ang2/anti-Dll4 binder according to SeqID No: 17 and bevacizumab.

10. Pharmaceutical combinations according to claim 3, comprising a dual anti-Ang2/anti-Dll4 binder according to SeqID No: 18 and bevacizumab.

11. Pharmaceutical composition comprising the pharmaceutical combination according claim 1, admixed with one or more pharmaceutically acceptable diluents and optionally further pharmaceutically acceptable agents.

12. Pharmaceutical composition according to claim 11 in the form of a combined preparation kit comprising
(i) a first compartment containing a first pharmaceutical composition comprising a dual anti-Ang2/anti-Dll4 binder, wherein the dual anti-Ang2/anti-Dll4 binders are selected from SeqID No: 1-20, and
(ii) a second compartment containing a second pharmaceutical composition comprising an anti-VEGF agent selected from bevacizumab, pegaptanib, ranibizumab, aflibercept and PRS-050, and optionally
(iii) a third compartment containing one or more pharmaceutical composition(s) comprising one or more additional anti-neoplastic agent(s).

13. A method of treating a disease comprising administering a pharmaceutical combination or a pharmaceutical composition thereof, according to claim 1, to a patient in need thereof.

14. The method of claim 13 wherein the disease is cancer.

15. The method of claim 14, wherein the cancer is selected from non-small cell lung cancer, renal cell carcinoma, ovarian cancer, breast cancer, colorectal cancer, and pancreatic cancer.

16. A method of treating cancer, comprising administering Dual anti-Ang2/anti-Dll4 binders in combination with anti-VEGF agents to a patient in need thereof.

17. A method of treatment of cancer, comprising administration of a therapeutically effective amount of a dual anti-Ang2/anti-Dll4 binder to a patient in need thereof, and furthermore comprising administration of a therapeutically effective amount of an anti-VEGF agent to the same patient within 72 hours before or after administration of said dual anti-Ang2/anti-Dll4 binder.

18. The method of claim 17, wherein administration of the anti-VEGF agent is done within 36 hours, preferably 24 hours, preferably 12 hours, preferably 6 hours, preferably 3 hours, preferably 2 hours, preferably 1 hour, preferably 30 minutes before or after administration of said dual anti-Ang2/anti-Dll4 binder.

19. The method of claim 17, wherein administration of the anti-VEGF agent is done simultaneously with the administration of said dual anti-Ang2/anti-Dll4 binder.
